(19) United States
(12) Patent Application Publication
(10) Pub. No.: US 2007/0122404 A1
(43) Pub. Date: May 31, 2007

(54) Humanized immunoglobulin reactive with alpha4beta7 integrin

(76) Inventor: Theresa L. O'Keefe, Waltham, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/599,151

(22) Filed: Nov. 14, 2006

Related U.S. Application Data
(60) Provisional application No. 60/737,582, filed on Nov. 17, 2005.

(51) Int. Cl. A61K 39/395; C07H 21/04; C12P 21/06; C12N 5/06; C07K 16/44

(52) U.S. Cl. 424/141.1; 530/388.15; 435/69.1; 435/320.1; 435/328; 536/23.53

(57) ABSTRACT

The present invention relates to a humanized immunoglobulin that has binding specificity for α4β7 integrin and comprises the complementarity determining regions (CDRs) of mouse Act-1 antibody, and to the humanized light chain of the humanized immunoglobulin. The present invention further relates to a humanized immunoglobulin light chain. The invention also relates to isolated nucleic acids, recombinant vectors and host cells that comprise a sequence which encodes a humanized immunoglobulin or immunoglobulin light chain, and to a method of preparing a humanized immunoglobulin. The humanized immunoglobulins can be used in therapeutic applications, for example to control lymphocyte infiltration to mucosal tissue or to treat inflammatory bowel disease.

### RELATED APPLICATION

This application claims the benefit of U.S. Provisional Application No. 60/737,582, filed on Nov. 17, 2005. The entire teachings of the above application is incorporated herein by reference.

### BACKGROUND OF THE INVENTION

Integrin receptors are important for regulating both lymphocyte recirculation and recruitment to sites of inflammation (Carlos, T. M. and Harlan, J. M., Blood, 84:2068-2101 (1994)). The human α4β7 integrin has several ligands, one of which is the mucosal vascular addressin MAdCAM-1 (Berlin, C., et al., Cell 74: 185-195 (1993); Erle, D. J., et al., J. Immunol. 153:517-528 (1994)), which is expressed on high endothelial venules in mesenteric lymph nodes and Peyer's patches (Streeter, P. R., et al., Nature 331:41-46 (1998)). As such, the α4β7 integrin acts as a homing receptor that mediates lymphocyte migration to intestinal mucosal lymphoid tissue (Schweighoffer, T., et al., J. Immunol. 151: 717-729 (1993)). In addition, the α4β7 integrin interacts with fibronectin and vascular cell adhesion molecule-1 (VCAM-1)

Inflammatory bowel disease (IBD), such as ulcerative colitis and Crohn's disease, for example, can be a debilitating and progressive disease involving inflammation of the gastrointestinal tract. IBD treatments have included anti-inflammatory drugs (such as, corticosteroids and sulfasalazine), immunosuppressive drugs (such as, 6-mercaptopurine, cyclosporine and azathioprine) and surgery (such as, colectomy). Podolsky, New Engl. J. Med., 325:928-937 (1991) and Podolsky, New Engl. J. Med., 325:1008-1016 (1991).

Antibodies against human α4β7 integrin, such as murine monoclonal antibody Act-1 (mAb Act-1), interfere with α4β7 integrin binding to mucosal addressin cell adhesion molecule-1 (MAdCAM-1) present on high endothelial venules in mucosal lymph nodes. Act-1 was originally isolated by Lazarovits, A. I., et al., J. Immunol. 133:1857-1862 (1984), from mice immunized with human tetanus toxoid-specific T lymphocytes and was reported to be a mouse IgG1/κ antibody. More recent analysis of the antibody by Schweighoffer, T., et al., J. Immunol. 151:717-729 (1993) demonstrated that it can bind to a subset of human memory CD4+ T lymphocytes which selectively express the α4β7 integrin. However, a serious problem with using murine antibodies for therapeutic applications in humans is that they are highly immuogenic in humans and quickly induce a human anti-murine antibody response (HAMA), which reduces the efficacy of the mouse antibody in patients and can prevent continued administration. The HAMA response results in rapid clearance of the mouse antibody, severely limiting any therapeutic benefit.

Thus, a need exists for improved therapeutic agents and approaches to inhibit cellular adhesion mediated through α4β7 integrin, for example, for the treatment of inflammatory bowel disease.

### SUMMARY OF THE INVENTION

The invention relates to a humanized immunoglobulin that has binding specificity for α4β7 integrin and comprises the complementarity determining regions (CDRs) of mouse Act-1 antibody, and relates to the humanized light chain of the humanized immunoglobulin. The humanized light chain of the invention has an amino acid sequence that is different from other humanized light chains that comprise the light chain CDRs of murine Act-1 antibody. The amino acid sequence of the humanized light chain of the invention is more human in nature (i.e., contains fewer amino acids that are not of human origin), and provides advantages over other antibodies that comprise the CDRs of Act-1.

The humanized immunoglobulin preferably comprises a humanized heavy chain and a humanized light chain. The amino acid sequence of the mature humanized heavy chain (e.g., after processing to remove the signal peptide) comprises the amino acid sequence of amino acid residues 20-470 of SEQ ID NO:2, and the amino acid sequence of the mature humanized light chain (e.g., after processing to remove the signal peptide) comprises the amino acid sequence of amino acid residues 20-238 of SEQ ID NO:4. For example, the humanized heavy chain can be encoded by a nucleic acid comprising the nucleotide sequence of nucleotides 87-1433 of SEQ ID NO:1, or comprising the nucleotide sequence of SEQ ID NO:1 or the open reading frame of SEQ ID NO:1, and the humanized light chain can be encoded by a nucleic acid comprising the nucleotide sequence of nucleotides 81-737 of SEQ ID NO:3, or comprising the nucleotide sequence of SEQ ID NO:3 or the open reading frame of SEQ ID NO:3. In some embodiments, the humanized immunoglobulin comprises a detectable label.

The invention also relates to the humanized light chain of the humanized immunoglobulin of the invention. The humanized light chain comprises the CDRs of murine Act-1 antibody. In one embodiment, the humanized immunoglobulin light chain comprises the amino acid sequence of amino acid residues 20-238 of SEQ ID NO:4. For example, the humanized light chain can be encoded by a nucleic acid comprising the nucleotide sequence of nucleotides 81-737 of SEQ ID NO:3, or comprising the nucleotide sequence of SEQ ID NO:3.

The invention also relates to isolated nucleic acid molecules that encode the humanized immunoglobulin or humanized light chain of the invention. In some embodiments, the invention is an isolated nucleic acid molecule encoding a humanized immunoglobulin heavy chain and a humanized immunoglobulin light chain which associate together to form a humanized immunoglobulin that has binding specificity for α4β7 integrin, wherein the humanized immunoglobulin heavy chain comprises the amino acid sequence of amino acid residues 20-470 of SEQ ID NO:2, and the humanized immunoglobulin light chain comprises the amino acid sequence of amino acid residues 20-238 of SEQ ID NO:4. In some embodiments, the isolated nucleic acid molecule encodes a humanized immunoglobulin heavy chain and a humanized immunoglobulin light chain which each comprise a signal peptide. For example, in some embodiments, the isolated nucleic acid molecule encodes a humanized immunoglobulin heavy chain that comprises the amino acid sequence of SEQ ID NO:2, and a humanized immunoglobulin light chain that comprises the amino acid sequence of SEQ ID NO:4.

In other embodiments, the invention is an isolated nucleic acid encoding a humanized immunoglobulin that has binding specificity for α4β7 integrin. In such embodiments, the isolated nucleic acid can comprise a first nucleotide sequence that encodes a humanized heavy chain and a second nucleotide sequence that encodes a humanized light chain, wherein the first nucleotide sequence comprises the nucleotide sequence of nucleotides 87-1433 of SEQ ID NO:1, and the second nucleotide sequence comprises the nucleotide sequence of nucleotides 81-737 of SEQ ID NO:3. In some embodiments, the first nucleotide sequence and the second nucleotide sequence each further comprise a nucleotide sequence that encodes a signal peptide. For example, the isolated nucleic acid molecule can comprise a first nucleotide sequence that comprises the nucleotide sequence of SEQ ID NO:1, and a second nucleotide sequence that comprises the nucleotide sequence of SEQ ID NO:3.

The invention also relates to an isolated nucleic acid molecule encoding the humanized light chain of the invention. In one embodiment, the isolated nucleic acid molecule encodes a humanized immunoglobulin light chain that comprises the amino acid sequence of amino acid residues 20-238 of SEQ ID NO:4. In some embodiments, the isolated nucleic acid molecule encodes a humanized immunoglobulin light chain that comprises a signal peptide. For example, the isolated nucleic acid molecule of the invention can encode a humanized immunoglobulin light chain that comprises the amino acid sequence of SEQ ID NO:4. In other embodiments, the isolated nucleic acid molecule comprises the nucleotide sequence of nucleotides 81-737 of SEQ ID NO: 3, and optionally comprises an additional nucleotide sequence that encodes a signal peptide. For example, the isolated nucleic acid molecule can comprise the nucleotide sequence of SEQ ID NO:3.

The invention also relates to recombinant vectors (e.g., expression vectors, mammalian cell expression vectors) that comprise a nucleic acid encoding the humanized immunoglobulin (humanized light chain and humanized heavy chain) or the humanized light chain of the invention. In some embodiments, the invention is a recombinant vector comprising a nucleic acid encoding a humanized immunoglobulin light chain that comprises the amino acid sequence of amino acid residues 20-238 of SEQ ID NO:4, and that optionally further comprises a signal peptide. For example, the recombinant vector can comprise the nucleotide sequence of nucleotides 81-737 of SEQ ID NO:3, or comprise the nucleotide sequence of SEQ ID NO:3. In some embodiments, the recombinant vector that comprises a nucleic acid encoding a humanized immunoglobulin light chain (e.g., a humanized immunoglobulin light chain that comprises the amino acid sequence of amino acid residues 20-238 of SEQ ID NO:4) further comprises a second nucleic acid encoding a humanized heavy chain comprising the amino acid sequence of amino acid residues 20-470 of SEQ ID NO:2. Optionally, the humanized heavy chain encoded by the second nucleic acid can further comprise a signal peptide. For example, the second nucleic acid can comprise the nucleotide sequence of nucleotides 87-1433 of SEQ ID NO:1, or comprises the nucleotide sequence of SEQ ID NO:1.

In other embodiments, the recombinant vector comprises a nucleic acid encoding a humanized immunoglobulin heavy chain and a humanized immunoglobulin light chain, wherein the humanized immunoglobulin heavy chain and the humanized immunoglobulin light chain associate together to form a humanized immunoglobulin that has binding specificity for α4β7 integrin. Preferably, the humanized immunoglobulin heavy chain comprises the amino acid sequence of amino acid residues 20-470 of SEQ ID NO:2, and the humanized immunoglobulin light chain comprises the amino acid sequence of amino acid residues 20-238 of SEQ ID NO:4. In certain embodiments, the humanized immunoglobulin heavy chain and the humanized immunoglobulin light chain each further comprise a signal peptide. For example, the isolated nucleic acid molecule can encode a humanized immunoglobulin heavy chain that comprises the amino acid sequence of SEQ ID NO:2, and a humanized immunoglobulin light chain that comprises the amino acid sequence of SEQ ID NO:4.

In other embodiments, the recombinant vector comprises a nucleic acid encoding a humanized immunoglobulin that has binding specificity for α4β7 integrin, wherein the isolated nucleic acid comprises a first nucleotide sequence that encodes a humanized heavy chain and a second nucleotide sequence that encodes a humanized light chain. Preferably, the first nucleotide sequence comprises the nucleotide sequence of nucleotides 87-1433 of SEQ ID NO:1, and the second nucleotide sequence comprises the nucleotide sequence of nucleotides 81-737 of SEQ ID NO:3. In certain embodiments, the first nucleotide sequence and the second nucleotide sequence each further comprise a nucleotide sequence that encodes a signal peptide. For example, the isolated nucleic acid molecule can comprise the nucleotide sequence of SEQ ID NO:1, and the nucleotide sequence of SEQ ID NO:3.

In particular embodiments, the recombinant vector of the invention is an expression vector, such as a mammalian cell expression vector.

The invention also relates to a host cell that comprises a recombinant nucleic acid encoding the humanized immunoglobulin (humanized light chain and humanized heavy chain) or the humanized light chain of the invention. For example, in some embodiments, the host cell comprises a recombinant vector (e.g., expression vector, mammalian expression vector) of the invention.

In some embodiments, the host cell comprises a recombinant nucleic acid encoding a humanized immunoglobulin light chain comprising the amino acid sequence of amino acid residues 20-238 of SEQ ID NO:4, and optionally comprising a signal peptide. For example, in some embodiments, the host cell comprises a recombinant nucleic acid that comprises the nucleotide sequence of nucleotides 81-737 of SEQ ID NO: 3, or comprises the nucleotide sequence of SEQ ID NO:3.

In some embodiments, the host cell comprises a recombinant nucleic acid encoding a humanized immunoglobulin light chain comprising the amino acid sequence of amino acid residues 20-238 of SEQ ID NO:4 and further comprises a second recombinant nucleic acid encoding a humanized heavy chain comprising the amino acid sequence of amino acid residues 20-470 of SEQ ID NO:2. In some embodiments, the second nucleic acid can encode a humanized heavy chain that further comprises a signal peptide. For example, in certain embodiments, the host cell comprises a second recombinant nucleic acid that comprises the nucleotide sequence of nucleotides 87-1433 of SEQ ID NO:1, or comprises the nucleotide sequence of SEQ ID NO:1.

In other embodiments, the host cell comprises a recombinant nucleic acid encoding a humanized immunoglobulin heavy chain and a humanized immunoglobulin light chain, wherein said humanized immunoglobulin heavy chain and the humanized immunoglobulin light chain associate together to form a humanized immunoglobulin that has binding specificity for α4β7 integrin. Preferably, the humanized immunoglobulin heavy chain comprises the amino acid sequence of amino acid residues 20-470 of SEQ ID NO:2, and the humanized immunoglobulin light chain comprises the amino acid sequence of amino acid residues 20-238 of SEQ ID NO:4. In certain embodiments, the humanized immunoglobulin heavy chain and the humanized immunoglobulin light chain each further comprise a signal peptide. For example, the host cell can comprise a recombinant nucleic acid encoding a humanized immunoglobulin heavy chain that comprises the amino acid sequence of SEQ ID NO:2, and a humanized immunoglobulin light chain that comprises the amino acid sequence of SEQ ID NO:4.

In other embodiments, the host cell comprises a recombinant nucleic acid encoding a humanized immunoglobulin that has binding specificity for α4β7 integrin, and comprises a first nucleotide sequence that encodes a humanized heavy chain and a second nucleotide sequence that encodes a humanized light chain. Preferably, the first nucleotide sequence comprises the nucleotide sequence of nucleotides 87-1433 of SEQ ID NO:1, and the second nucleotide sequence comprises the nucleotide sequence of nucleotides 81-737 of SEQ ID NO:3. In certain embodiments, the first nucleotide sequence and the second nucleotide sequence each further comprise a nucleotide sequence that encodes a signal peptide. For example, the host cell can comprise a first nucleotide sequence that comprises the nucleotide sequence of SEQ ID NO:1, and a second nucleotide sequence that comprises the nucleotide sequence of SEQ ID NO:3.

The invention also relates to a method of preparing a humanized immunoglobulin comprising maintaining a host cell of the invention (e.g., a host cell that contains one or more recombinant nucleic acids that encode a humanized immunoglobulin (e.g., a humanized light chain and a humanized heavy chain) of the invention) under conditions appropriate for expression of a humanized immunoglobulin, whereby humanized immunoglobulin chains are expressed and a humanized immunoglobulin is produced. In some embodiments, the method further comprises isolating the humanized immunoglobulin.

The invention further relates to a pharmaceutical composition comprising a humanized immunoglobulin or humanized light chain of the invention and a physiologically acceptable vehicle or carrier. In some embodiments, the pharmaceutical composition comprises multiple doses, and each dose comprises an effective amount of the humanized antibody or humanized light chain or antigen-binding fragment thereof. In other embodiments, the pharmaceutical composition is a unit dose composition that comprises an effective amount of the humanized antibody or humanized light chain.

The invention also relates to a method of inhibiting the interaction of a first cell bearing α4β7 with a second cell bearing a ligand thereof, comprising contacting the first cell with an effective amount of a humanized immunoglobulin or humanized light chain of the invention.

The invention also relates to a method of inhibiting leukocyte infiltration of mucosal tissue, comprising administering to a patient an effective amount a humanized immunoglobulin or humanized light chain of the invention.

The invention also relates to a method for treating a disease associated with leukocyte infiltration of tissues expressing the molecule MAdCAM-1, comprising administering to a patient in need thereof an effective amount of a humanized immunoglobulin or humanized light chain of the invention. For example, the disease can be mastitis, or chronic sinusitis. In some embodiments, the disease can be a disease associated with leukocyte infiltration of tissues as a result of binding of leukocytes expressing α4β7 integrin to gut-associated endothelium expressing the molecule MAdCAM-1. In particular embodiments, the disease is pancreatitis, insulin-dependent diabetes mellitus, cholecystitis, cholangitis, pericholangitis.

The invention also relates to a method for treating inflammatory bowel disease in a patient, comprising administering to the patient an effective amount of a humanized immunoglobulin or humanized light chain of the invention. In some embodiments, the inflammatory bowel disease is ulcerative colitis or Crohn's disease. In other embodiments, the inflammatory bowel disease is Celiac disease, enteropathy associated with seronegative arthropathies, microscopic or collagenous colitis, eosinophilic gastroenteritis, or pouchitis.

The invention also relates to immunoconjugates comprising a humanized immunoglobulin or an antigen-binding fragment thereof of the invention. In some aspects the immunoconjugate comprises a cytotoxic moiety (e.g., cytotoxic agent). For example, the cytotoxic moiety can comprise a radioisotope, a therapeutic agent (e.g., a chemotherapeutic agent, an antimetabolite, an alkylating agent, an anthracycline, an antibiotic, an anti-mitotic agent, a biological response modifier (e.g., a cytokine (e.g., an interleukin, an interferon, a tumor necrosis factor), a growth factor (e.g., a neurotrophic factor)), or a tumor activated pro-drug (e.g., an enzyme and/or enzyme activated compound). When the cytotoxic moiety of the immunoconjugate is a therapeutic agent, the agent can comprise a maytansine, an auristatin, a dolastatin, a duocarmycin, a cryptophycin, a taxol, a DNA alkylating agent, a calicheamicin, or a derivative of any of the foregoing. In some aspects the cytotoxic moiety comprises a maytansine selected from DM1 and DM4.

The invention also relates to a humanized immunoglobulin or humanized light chain of the invention for use in medicine, such as for use in therapy and/or diagnosis. The invention also relates to the use of a humanized immunoglobulin or humanized light chain of the invention for the manufacture of a medicament for treating a disease or disorder described herein (e.g., inflammatory bowel disease (e.g., ulcerative colitis, Crohn's disease)).

The invention also relates to a pharmaceutical composition for treating a disease or disorder described herein (e.g., inflammatory bowel disease (e.g., ulcerative colitis, Crohn's disease)), comprising a humanized immunoglobulin or humanized light chain of the invention as an active ingredient. In some embodiments, the pharmaceutical composition comprises one or more additional active ingredients, such as, an anti-inflammatory compound (e.g., sulfasalazine, a nonsteroidal anti-inflammatory compound, or steroidal anti-inflammatory compound).

### BRIEF DESCRIPTION OF THE DRAWINGS

FIG. 1 is an illustration of a nucleotide sequence (SEQ ID NO:1) encoding the heavy chain of a humanized immunoglobulin referred to herein as MLN02, and the deduced amino acid sequence of the heavy chain (SEQ ID NO:2). The nucleotide sequence contains cloning sites (lower case), Kozak sequence (upper case, nucleotides 18-23 of SEQ ID NO:1) and leader sequence (lower case, nucleotides 24-86 of SEQ ID NO:1) at the 5′ end of the heavy chain. The open reading frame of the nucleotide sequence is nucleotides 24-1433 of SEQ ID NO:1.

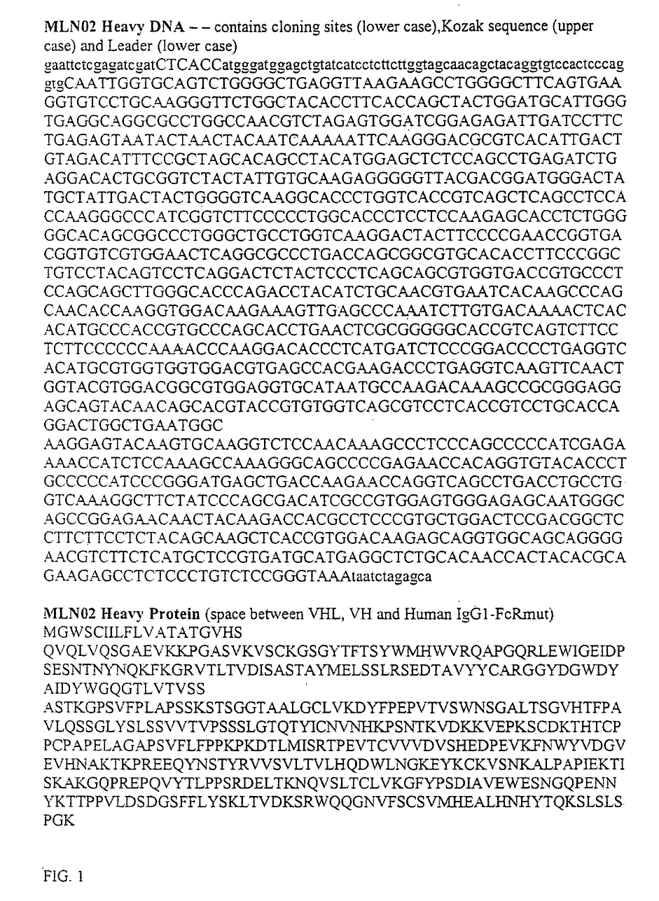

FIG. 2 is an illustration of a nucleotide sequence (SEQ ID NO:3) encoding the light chain of a humanized immunoglobulin referred to herein as MLN02, and the deduced amino acid sequence (SEQ ID NO: 4) of the light chain. The nucleotide sequence contains cloning sites (lower case), Kozak sequence (upper case, nucleotides 18-23 of SEQ ID NO:3) and leader sequence (lower case, nucleotides 24-80 of SEQ ID NO:3) at the 5′ end of the heavy chain. The open reading frame of the nucleotide sequence is nucleotides 24-737 of SEQ ID NO:3.

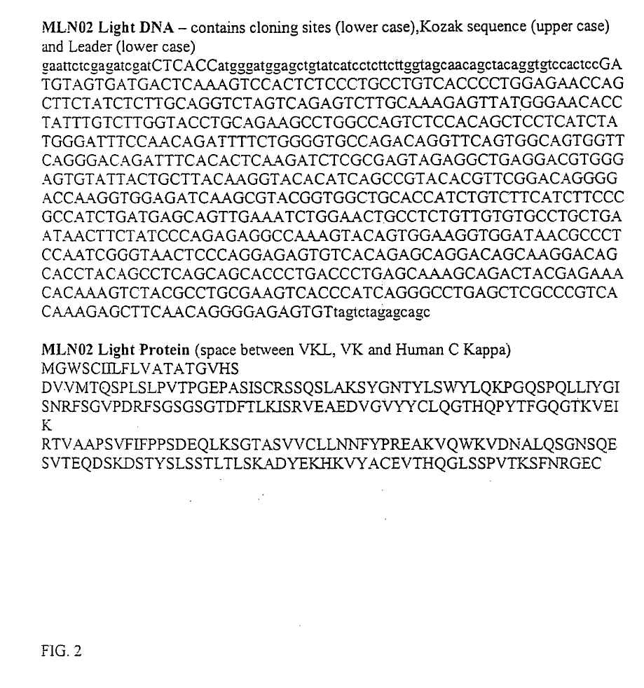

FIG. 3 is an alignment of the amino acid sequences of (A) the mature humanized light chain (amino acids 20-238 of SEQ ID NO:4) of the humanized immunoglobulin referred to herein as MLN02 and (B) the mature humanized light chain of the humanized immunoglobulin referred to herein as LDP-02 (SEQ ID NO:5). (Regarding LDP-02, see, WO 98/06248 and Feagan et al., N. Eng. J. Med. 352:2499-2507 (2005). Feagan et al. describe a clinical study of LDP-02, but in the article they refer to LDP-02 as MLN02.) The alignment illustrates that the amino acid sequences of the light chains of MLN02 and LDP-02 differ at positions 114 and 115 of the mature light chains.

FIG. 4 is an alignment of the amino acid sequences of (A) the human kappa light chain constant region (SEQ ID NO:6) and (B) the murine kappa light chain constant region (SEQ ID NO:7). The amino acid residues Thr and Val (which are present at positions 114 and 115 of the mature MLN02 light chain (amino acids 133 and 134 of SEQ ID NO:4)) are present in the constant region of the human kappa light chain, whereas the amino acid residues Ala and Asp (which are present at positions 114 and 115 of the mature LDP-02 light chain (SEQ ID NO:5)) are present in the constant region of the mouse kappa light chain. The alignment illustrates that the humanized light chain of MLN02 is more human than the light chain of LDP-02.

FIG. 5 is a map of vector pLKTOK38D (also referred to as pTOK38MLN02-TV), which encodes the humanized heavy chain and the humanized light chain of MLN02, and is suitable for producing MLN02 in CHO cells. (See, U.S. Patent Application Publication No. 2004/0033561 A1 which discloses pLKTOK38. pLKTOK38D is a variant of pLKTOK38 in which the restriction sites indicated on the map flank the sequence encoding the light chain variable region.)

FIG. 6 is a graph and table illustrating the results of adhesion assays that demonstrated that MLN02 and LDP-02 inhibited the adhesion of α4β7-expressing RPMI-8866 cells to immobilized MAdCAM-1 with similar potency. Two preparations of MLN02 that were produced by different CHO cell clones (MLN02 CHO10.21 (CHO10.21) and MLN02 CHO24.9A (CHO24.9A)) and LDP-02 produced by NS0 cells (LDP-02 NS0 (NS06868.4)) were studied using the assay.

FIGS. 7A and 7B are graphs showing the qualitative amount of LDP-02 (FIG. 7A) or MLN02 (FIG. 7B) that was bound to α4β7 on the surface of CD4⁺ cells in cynomolgus monkeys that were administered LDP-02 (FIG. 7A) or MLN02 (FIG. 7B). Blood samples were obtained from cynomolgus monkeys prior to administering doses of LDP-02 or MLN-02, and the amount of α4β7 sites on the cells in the blood that were bound by LDP-02 or MLN-02 was determined. The results show good saturation of α4β7 by LDP-02 and by MLN-02. One animal (2003) that was administered MLN02 developed a PAHA response, and showed reduced α4β7 saturation at the last time point assayed.

FIGS. 8A and 8B are graphs showing the relative number of α4β7 sites on the surface of CD4⁺ cells in cynomolgus monkeys that were administered LDP-02 (FIG. 7A) or MLN02 (FIG. 7B) that were not bound by LDP-02 (FIG. 7A) or MLN02 (FIG. 7B). Blood samples were obtained from cynomolgus monkeys prior to administering doses of LDP-02 or MLN-02, and the amount of α4β7 sites on the cells in the blood that were not bound by LDP-02 or MLN-02 was determined. The results, including the amount of free α4β7 sites on CD4⁺ cells in a blood sample obtained prior to commencing the study, show that the relative amount of free α4β7 sites dramatically decreased following administration LDP-02 or MLN-02. These results demonstrate that good saturation of α4β7 was achieved by LDP-02 and by MLN-02. One animal (2003) that was administered MLN02 developed a PAHA response, and showed reduced α4β7 saturation at the last time point assayed.

FIGS. 9A and 9B are graphs in which the data presented in FIGS. 8A and 8B was replotted without the pre-study data. These graphs provide a more detailed view of the relative amounts of free α4β7 sites on CD4⁺ cells during the course of the study. These results demonstrate that good saturation of α4β7 was achieved by LDP-02 and by MLN-02. One animal (2003) that was administered MLN02 developed a PAHA response, and showed reduced α4β7 saturation at the last time point assayed.

FIGS. 10A and 10B are graphs showing the qualitative amount of LDP-02 (FIG. 10A) or MLN02 (FIG. 10B) that was bound to α4β7 on the surface of CD8⁺ cells in cynomolgus monkeys that were administered LDP-02 (FIG. 10A) or MLN02 (FIG. 10B). Blood samples were obtained from cynomolgus monkeys prior to administering doses of LDP-02 or MLN-02, and the amount of α4β7 sites on the cells in the blood that were bound by LDP-02 or MLN-02 was determined. The results show good saturation of α4β7 by LDP-02 and by MLN-02. One animal (2003) that was administered MLN02 developed a PAHA response, and showed reduced α4β7 saturation at the last time point assayed.

FIGS. 11A and 11B are graphs showing the relative number of α4β7 sites on the surface of CD8⁺ cells in cynomolgus monkeys that were administered LDP-02 (FIG. 11A) or MLN02 (FIG. 11B) that were not bound by LDP-02 (FIG. 11A) or MLN02 (FIG. 11B). Blood samples were obtained from cynomolgus monkeys prior to administering doses of LDP-02 or MLN-02, and the amount of α4β7 sites on the cells in the blood that were not bound by LDP-02 or MLN-02 was determined. The results, including the amount of free α4β7 sites on CD4⁺ cells in a blood sample obtained prior to commencing the study, show that the relative amount of free α4β7 sites dramatically decreased following administration LDP-02 or MLN-02. These results demonstrate that good saturation of α4β7 was achieved by LDP-02 and by MLN-02. One animal (2003) that was administered MLN02 developed a PAHA response, and showed reduced α4β7 saturation at the last time point assayed.

FIGS. 12A and 12B are graphs in which the data presented in FIGS. 11A and 11B was replotted without the pre-study data. These graphs provide a more detailed view of the relative amounts of free α4β7 sites on CD4⁺ cells during the course of the study. These results demonstrate that good saturation of α4β7 was achieved by LDP-02 and by MLN-02. One animal (2003) that was administered MLN02 developed a PAHA response, and showed reduced α4β7 saturation at the last time point assayed.

### DETAILED DESCRIPTION OF THE INVENTION

The term “immunoglobulin” as used herein refers to whole antibodies and antigen-binding fragments thereof. Antigen-binding fragments of antibodies include, for example, single chain antibodies, Fv fragments, Fab fragments, Fab′ fragments and F(ab′)₂ fragments. Such fragments can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH, domain and hinge region of the heavy chain.

The term “humanized immunoglobulin” as used herein refers to an immunoglobulin containing one or more humanized immunoglobulin chains that comprise the heavy chain CDRs (CDR1, CDR2 and CDR3) and light chain CDRs (CDR1, CDR2 and CDR3) of murine Act-1 antibody, and framework and constant regions derived from a light and/or heavy chain of human origin (e.g., CDR-grafted antibodies with or without framework changes). The humanized immunoglobulin of the invention comprises at least amino acid residues 20-134 of the humanized light chain sequence of SEQ ID NO:4. CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1. See also, Ladner et al., U.S. Pat. No. 4,946,778; Huston, U.S. Pat. No. 5,476,786; and Bird, R. E. et al., Science, 242: 423-426 (1988)), regarding single chain antibodies.

“Antigen-binding fragments” of the humanized immunoglobulin of the invention comprise at least amino acid residues 20-134 of the humanized light chain sequence of SEQ ID NO:4. Examples of such antigen-binding fragments include Fab fragments, Fab′ fragments and F(ab′)₂ fragments of a humanized immunoglobulin that contains a humanized light chain comprising amino acid residues 20-238 of SEQ ID NO:4, and a humanized heavy chain comprising amino acid residues 20-470 of SEQ ID NO:2. Antigen-binding fragments of the humanized immunoglobulin of the invention can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a recombinant construct encoding the heavy chain of an F(ab′)₂ fragment can be designed to include DNA sequences encoding the CH_{I} domain and hinge region of the heavy chain. Preferred antigen-binding fragments inhibit binding of α4β7 to one or more of its ligands (e.g. the mucosal addressin MAdCAM-1, fibronectin)

As described herein, a humanized immunoglobulin comprising the CDRs of the murine Act-1 antibody has been produced. The humanized immunoglobulin comprises a humanized light chain that has an amino acid sequence which differs from the amino acid sequence of other humanized versions of murine Act-1 antibody (e.g., the light chain of LDP-02). The amino acid sequence of the humanized light chain of the invention is more human in character and, thus, is expected to be even less immunogenic in human beings. In addition, as described herein, the humanized immunoglobulin of the invention is more compact than LDP-02, a property that is associated with increased stability, resistance to degradation and aggregation, and solubility.

The invention relates to the humanized immunoglobulin described herein, antigen-binding fragments of the humanized immunoglobulin, and the humanized light chain of the humanized immunoglobulin and fragments thereof. The invention relates to mature humanized immunoglobulin, such as the humanized immunoglobulin following processing to remove the heavy and light signal peptides and/or to the glycosylated immunoglobulin. The invention also relates to immature or precursor protein, such as a humanized light chain comprising a signal peptide. The invention also relates to nucleic acid molecules (e.g., vectors) that encode both immature and mature protein, to host cells that comprise such nucleic acids and to methods of producing immature and mature proteins.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by Kabat et al. (Kabat, E. A. et al., Sequences of Proteins of Immunological Interest, Fifth Edition, U.S. Department of Health and Human Services, U.S. Government Printing Office (1991)).

Human immunoglobulins can be divided into classes and subclasses, depending on the isotype of the heavy chain. The classes include IgG, IgM, IgA, IgD and IgE, in which the heavy chains are of the gamma (γ), mu (μ), alpha (α), delta (δ) or epsilon (ε) type, respectively. Subclasses include IgG1, IgG2, IgG3, IgG4, IgA1 and IgA2, in which the heavy chains are of the γ1, γ2, γ3, γ4, α1 and α2 type, respectively. Human immunoglobulin molecules of a selected class or subclass may contain either a kappa (κ) or lambda (λ) light chain. See e.g., Cellular and Molecular Immunology, Wonsiewicz, M. J., Ed., Chapter 45, pp. 41-50, W. B. Saunders Co., Philadelphia, Pa. (1991); Nisonoff, A., Introduction to Molecular Immunology, 2nd Ed., Chapter 4, pp. 45-65, Sinauer Associates, Inc., Sunderland, Mass. (1984).

The CDRs of the humanized immunoglobulin (the nonhuman portion) are from the murine Act-1 monoclonal antibody (Lazarovits, A. I. et al., J. Immunol., 133(4): 1857-1862 (1984)). The murine Act-1 monoclonal antibody has binding specificity for the α4β7 integrin heterodimer, and does not bind other integrins that contain the α4 chain or β7 chain. (See, Schweighoffer et al., J. Immunol., 151:717-729 (1993).) Accordingly, the humanized immunoglobulin of the invention has better target selectivity than other antibodies and humanized immunoglobulins that bind the α4 chain, such as natalizumab (Biogen Idec; Cambridge, Mass.) which binds α4β1 integrin and α4β7 integrin.

Preferably, the humanized immunoglobulins bind α4β7 integrin with an affinity similar to or better than that of murine Act-1 antibody. In a preferred embodiment, the humanized immunoglobulin of the present invention has the binding specificity of murine Act-1 antibody, (e.g., having specificity for α4β7 integrin, having the same or similar epitopic specificity) and/or inhibitory function (e.g., the ability to inhibit α4β7 dependent adhesion in vitro and/or in vivo, such as the ability to inhibit α4β7 integrin binding to MAdCAM-1 in vitro and/or in vivo, or the ability to inhibit the binding of a cell bearing α4β7 integrin to a ligand thereof (e.g., a cell bearing MAdCAM-1)). Thus, preferred humanized immunoglobulins can have the binding specificity of the murine Act-1 antibody, the epitopic specificity of the murine Act-1 antibody (e.g., can compete with murine Act-1, or humanized Act-1 (e.g., LDP-02) for binding to α4β7 (e.g., on a cell bearing α4β7 integrin)), and inhibitory function of murine Act-1 antibody. Preferably, the humanized immunoglobulins can bind α4β7 integrin with an affinity of at least about 10⁷M⁻¹, preferably at least about 10⁸ M⁻¹, and more preferably at least about 10⁹M⁻¹.

Murine ACT-1 Hybridoma cell line, which produces the murine Act-1 monoclonal antibody was deposited under the provisions of the Budapest Treaty on Aug. 22, 2001, on behalf of Millennium Pharmaceuticals, Inc., 75 Sidney Street, Cambridge, Mass. 02139, U.S.A., at the American Type Culture Collection, 10801 University Boulevard, Manassas, Va. 20110-2209, U.S.A., under Accession No. PTA-3663.

The portion of the humanized immunoglobulin or immunoglobulin chain which is of human origin (framework and constant regions) can be derived from any suitable human immunoglobulin or immunoglobulin chain. For example, a human constant region or portion thereof can be derived from the κ or λ light chains, and/or the γ (e.g., γ1, γ2, γ3, γ4), μ, α(e.g., α1, α2), δ or ε heavy chains of human antibodies, including allelic variants. A particular constant region (e.g., IgG1), variant or portion thereof can be selected in order to tailor effector function. For example, a mutated constant region (variant) can be incorporated into a fusion protein to minimize binding to Fc receptors and/or ability to fix complement. (see e.g. Winter et al., GB 2,209,757 B; Morrison et al., WO 89/07142; Morgan et al., WO 94/29351, Dec. 22, 1994).

Human framework regions and constant regions (e.g., of the light chain variable region) are preferably derived from a human antibody variable region having sequence similarity to the analogous or equivalent region (e.g., light chain variable region) of the antigen binding region of the donor (murine Act-1 antibody). Other sources of framework regions for portions of human origin of a humanized immunoglobulin include human variable consensus sequences (see e.g. Kettleborough, C. A. et al., Protein Engineering 4:773-783 15 (1991); Carter et al., WO 94/04679)). For example, the sequence of the antibody or variable region used to obtain the nonhuman portion can be compared to human sequences as described in Kabat, E. A., et al., Sequences of Proteins of Immunological Interest, Fifth Edition, U.S. Department of Health and Human Services, U.S. Government Printing Office (1991).

The immunoglobulin portions of nonhuman and human origin for use in the present invention have sequences identical to immunoglobulins or immunoglobulin portions from which they are derived or to variants thereof. Such variants include mutants differing by the addition, deletion, or substitution of one or more residues. As indicated above, the humanized immunoglobulin of the invention comprises the CDRs of the murine Act-1 antibody. Changes in the framework region, such as those which substitute a residue of the framework region of human origin with a residue from the corresponding position of the donor antibody, can be made. One or more mutations in the framework region can be made, including deletions, insertions and substitutions of one or more amino acids. If desired, framework mutations can be included in a humanized antibody or chain, and sites for mutation can be selected using any suitable method, for example as described in WO 98/06248, the entire teachings of which are incorporated by reference.

The binding function of a humanized immunoglobulin having binding specificity for α4β7 integrin can be detected using any suitable method, for example using assays which monitor formation of a complex between humanized immunoglobulin and α4β7 integrin (e.g., a membrane fraction comprising α4β7 integrin, on a cell bearing α4β7 integrin, such as a human lymphocyte (e.g., a lymphocyte of the CD4+α4^{hi}, β1^{lo} subset), human lymphocyte cell line or recombinant host cell comprising nucleic acid encoding α4 and/or β7 (which expresses α4β7 integrin)).

Binding and/or adhesion assays or other suitable methods can also be used in procedures for the identification and/or isolation of humanized immunoglobulins (e.g., from a library) with the requisite specificity (e.g., an assay which monitors adhesion between a cell bearing an α4β7 integrin and a ligand thereof (e.g., a second cell expressing MAdCAM, a MAdCAM-Ig fusion protein, or other suitable methods)).

The immunoglobulin portions of nonhuman and human origin that can be used in the present invention include light chains, heavy chains and portions of light and heavy chains. These immunoglobulin portions can be obtained or derived from immunoglobulins (e.g., by de novo synthesis of a portion), or nucleic acids encoding an immunoglobulin or chain thereof having the desired property (e.g., binds α4β7 integrin, sequence similarity) can be produced and expressed. Humanized immunoglobulins comprising the desired portions (e.g., antigen binding region, CDR, FR, C region) of human and nonhuman origin can be produced using synthetic and/or recombinant nucleic acids to prepare constructs (e.g., cDNA) encoding the desired humanized chain. To prepare a portion of a chain, one or more stop codons can be introduced at the desired position. For example, nucleic acid (e.g., DNA) sequences coding for new humanized variable regions can be constructed using PCR mutagenesis methods to alter existing DNA sequences (see e.g., Kamman, M., et al., Nucl. Acids Res. 17:5404 (1989)). PCR primers coding for the new CDRs can be hybridized to a DNA template of a previously humanized variable region which is based on the same, or a very similar, human variable region (Sato, K., et al., Cancer Research 53:851-856 (1993)). If a similar DNA sequence is not available for use as a template, a nucleic acid comprising a sequence encoding a variable region sequence can be constructed from synthetic oligonucleotides (see, e.g., Kolbinger, F., Protein Engineering 8:971-980 (1993)). A sequence encoding a signal peptide can also be incorporated into the nucleic acid (e.g., on synthesis, upon insertion into a vector). If the natural signal peptide sequence is unavailable, a signal peptide sequence from another antibody can be used (see, e.g., Kettleborough, C. A., Protein Engineering 4:773-783 (1991)). Using these methods, methods described herein or other suitable methods, variants can be readily produced.

The invention relates to a humanized immunoglobulin that has binding specificity for α4β7 integrin and comprises a humanized heavy chain and a humanized light chain. In one embodiment, the humanized immunoglobulin comprises a mature humanized heavy chain comprising amino acid residues 20-470 of SEQ ID NO:2 and a mature humanized light chain comprising amino acid residues 20-238 of SEQ ID NO:4. For example, in one embodiment, the humanized heavy chain and humanized light chain have been processed to remove the signal peptide.

In another embodiment, the humanized immunoglobulin comprises an immature humanized heavy chain comprising SEQ ID NO:2 and an immature humanized light chain comprising SEQ ID NO:4. In some embodiments, the humanized immunoglobulin comprises a detectable label.

The invention also relates to a humanized immunoglobulin as described herein that is produced by expression in a CHO cell line, such as CHO DG44.

In some embodiments, the humanized immunoglobulin is glycosylated and comprises predominantly agalactosylated glycoforms (e.g., G0F glycoforms). In particular embodiments, at least about 40%, at least about 45%, at least about 50%, at least about 55%, at least about 60%, at least about 65%, at least about 70%, at least about 75%, or at least about 80% of the glycoforms present on the humanized immunoglobulin are G0 glycoforms.

Preferably, the humanized immunoglobulin is stable for at least about 6 months, or at least about 12 months, or at least about 18 months, or at least about 24 months, or at least about 30 months. Stability is preferably determined by maintaining a 10 mg/mL solution of humanized immunoglobulin in an aqueous buffer (e.g., 20 mM sodium citrate pH 6.0 containing 125 mM sodium chloride) at 40° C. for a period of at least about 6 months, and then assessing the degree of aggregation or degradation. The humanized immunoglobulin is stable if less than about 10%, or less than about 9%, or less than about 8%, or less than about 7%, or less than about 6%, or less than about 5%, or less than about 4%, or less than about 3%, or less than about 2%, or less than about 1%, or substantially none of the humanized antibody in the solution aggregates or is degraded.

The invention also relates to a humanized immunoglobulin light chain of the humanized immunoglobulin described herein. In one embodiment, the humanized immunoglobulin light chain comprises the amino acid sequence of amino acid residues 20-238 of SEQ ID NO:4. If desired, the humanized immunoglobulin light chain can further comprise a signal peptide. For example, the humanized immunoglobulin light chain can have the amino acid sequence of SEQ ID NO:4.

The invention also relates to antigen-binding fragments of the humanized immunoglobulin described herein, and to fragment of the humanized light chain that comprise at least amino acid residues 20-134 of SEQ ID NO:4.

If desired, for example for diagnostic purposes (e.g. imaging), the humanized immunoglobulin or antigen-binding fragment thereof can comprise a detectable label. Suitable detectable labels and methods for labeling a humanized immunoglobulin or antigen-binding fragment thereof are well known in the art. Suitable detectable labels include, for example, a radioisotope (e.g., as Indium-111, Technetium-99m or Iodine-131), positron emitting labels (e.g., Fluorine-19), paramagnetic ions (e.g., Gadlinium (III), Manganese (II)), an epitope label (tag), an affinity label (e.g., biotin, avidin), a spin label, an enzyme, a fluorescent group or a chemiluminescent group. When labels are not employed, complex formation (e.g., between humanized immunoglobulin and α4β7 integrin) can be determined by surface plasmon resonance or other suitable methods.

The invention also relates to immunoconjugates comprising the humanized antibody or antigen-binding fragment thereof of the invention and another therapeutic agent, such as a cytotoxic moiety. For example, MLN02 or antigen-binding fragment thereof can be coupled to a biological protein, a molecule of plant or bacterial origin (or derivative thereof), e.g., a maytansinoid (e.g., maytansinol, e.g., DM1, DM4), a taxane, a calicheamicin, a duocarmycin, or derivatives thereof. The maytansinoid can be, for example, maytansinol or a maytansinol analogue. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U.S. Pat. Nos. 5,208,020, and 6,333,410, the contents of which is incorporated herein by reference. Maytansinol can be coupled to antibodies using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2 iminothiolane, or S-acetylsuccinic anhydride. The taxane can be, for example, a taxol, taxotere, or novel taxane (see, e.g., WO 01/38318). The calicheamicin can be, for example, a bromo-complex calicheamicin (e.g., an alpha, beta or gamma bromo-complex), an iodo-complex calicheamicin (e.g., an alpha, beta or gamma iodo-complex), or analogs and mimics thereof. Bromo-complex calicheamicins include I1-BR, I2-BR, I3-BR, I4-BR, J1-BR, J2-BR and K1-BR. Iodo-complex calicheamicins include I1-I, I2-I, I3-I, J1-I, J2-I, L1-I and K1-BR. Calicheamicin and mutants, analogs and mimics thereof are described, for example, in U.S. Pat. Nos. 4,970,198; 5,264,586; 5,550,246; 5,712,374, and 5,714,586, the contents of each of which are incorporated herein by reference. Duocarmycin analogs (e.g., KW-2189, DC88, DC89 CBI-TMI), and derivatives thereof are described, for example, in U.S. Pat. No. 5,070,092, U.S. Pat. No. 5,187,186, U.S. Pat. No. 5,641,780, U.S. Pat. No. 5,641,780, U.S. Pat. No. 4,923,990, and U.S. Pat. No. 5,101,038, the contents of each of which are incorporated herein by reference.

Examples of other therapeutic agents include, but are not limited to, cytochalasin B, gramicidin D, ethidium bromide, emetine, etoposide, tenoposide, colchicin, dihydroxy anthracin dione, mitoxantrone, 1-dehydrotestosterone, glucocorticoids, procaine, tetracaine, lidocaine, propranolol, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065 (see, U.S. Pat. Nos. 5,475,092, 5,585,499, and 5,846,545), melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, mitomycin, puromycin anthramycin (AMC)), duocarmycin and analogs or derivatives thereof, and anti-mitotic agents (e.g., vincristine, vinblastine, taxol, auristatins (e.g., auristatin E) and maytansinoids, and analogs or homologs thereof.

The immunoconjugates of the invention can be used for modifying a given biological response. The therapeutic agent is not to be construed as limited to classical chemical therapeutic agents. For example, the therapeutic agent may be a protein or polypeptide possessing a desired biological activity. Such proteins may include, for example, a toxin such as abrin, ricin A, pseudomonas exotoxin, gelonin, diphtheria toxin, or a component thereof (e.g., a component of pseudomonas exotoxin is PE38); a protein such as tumor necrosis factor, interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL 6”), granulocyte macrophase colony stimulating factor (“GM-CSF”), granulocyte colony stimulating factor (“G-CSF”), or other growth factors. Similarly, the therapeutic agent can be a viral particle, e.g., a recombinant viral particle, that is conjugated (e.g., via a chemicallinker) or fused (e.g., via a viral coat protein) to an anti-OV064 antibody of the invention. Introduction of the viral nucleic acid molecules, e.g., recombinant viral nucleic acid molecules, into cells, e.g., cancer cells that express α4β7 can occur following binding and endocytosis of the MLN02/viral particle conjugate or fusion.

Therapeutically active radionuclides can also be coupled to MLN02 or antigen-binding fragments thereof. Radionuclides (e.g., radioactive isotopes) can be used in diagnostic or therapeutic applications. Radioactive isotopes that can be coupled to the MLN02, include but are not limited to alpha-, beta-, or gama-emitters, or beta- and gama-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³ Bi), indium (¹¹¹In), technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (131I or 125I), indium (¹¹¹In), technetium (⁹⁹mTc), phosphorus (³²P), carbon (¹⁴C), and tritium (³H), or one or more of the therapeutic isotopes listed above.

Nucleic Acids and Recombinant Vectors

The present invention also relates to isolated and/or recombinant (including, e.g., essentially pure) nucleic acids comprising sequences which encode a humanized immunoglobulin or humanized immunoglobulin light chain of the present invention.

Nucleic acids referred to herein as “isolated” are nucleic acids which have been separated away from the nucleic acids of the genomic DNA or cellular RNA of their source of origin (e.g., as it exists in cells or in a mixture of nucleic acids such as a library), and include nucleic acids obtained by methods described herein or other suitable methods, including essentially pure nucleic acids, nucleic acids produced by chemical synthesis, by combinations of biological and chemical methods, and recombinant nucleic acids which are isolated (see e.g., Daugherty, B. L. et al., Nucleic Acids Res., 19(9): 2471-2476 (1991); Lewis, A. P. and J. S. Crowe, Gene, 101: 297-302 (1991)).

Nucleic acids referred to herein as “recombinant” are nucleic acids which have been produced by recombinant DNA methodology, including those nucleic acids that are generated by procedures which rely upon a method of artificial recombination, such as the polymerase chain reaction (PCR) and/or cloning into a vector using restriction enzymes. “Recombinant” nucleic acids are also those that result from recombination events that occur through the natural mechanisms of cells, but are selected for after the introduction to the cells of nucleic acids designed to allow and make probable a desired recombination event.

The present invention also relates more specifically to isolated and/or recombinant nucleic acids comprising a nucleotide sequence which encodes a humanized Act-1 immunoglobulin (i.e., a humanized immunoglobulin of the present invention in which the nonhuman portion is derived from the murine Act-1 monoclonal antibody) or light chain thereof.

Nucleic acids of the present invention can be used in the production of humanized immunoglobulins having binding specificity for α4β7 integrin. For example, a nucleic acid (e.g., DNA) encoding a humanized immunoglobulin of the present invention can be incorporated into a suitable construct (e.g., a recombinant vector) for further manipulation of sequences or for production of the encoded polypeptide in suitable host cells.

Constructs or vectors (e.g., expression vectors) suitable for the expression of a humanized immunoglobulin having binding specificity for α4β7 integrin are also provided. A variety of vectors are available, including vectors which are maintained in single copy or multiple copy, or which become integrated into the host cell chromosome. The constructs or vectors can be introduced into a suitable host cell, and cells which express a humanized immunoglobulin of the present invention, can be produced and maintained in culture.

Suitable expression vectors, for example mammalian cell expression vectors, can also contain a number of components, including, but not limited to one or more of the following: an origin of replication; a selectable marker gene; one or more expression control elements, such as a transcriptional control element (e.g., a promoter, an enhancer, terminator), and/or one or more translation signals; a signal sequence or leader sequence for membrane targeting or secretion. In a construct or vector, a signal peptide sequence can be provided by the construct or vector or other source. For example, the transcriptional and/or translational signals of an immunoglobulin can be used to direct expression.

A promoter can be provided for expression in a suitable host cell. Promoters can be constitutive or inducible. For example, a promoter can be operably linked to a nucleic acid encoding a humanized immunoglobulin or immunoglobulin chain, such that it directs expression of the encoded polypeptide. A variety of suitable promoters for prokaryotic (e.g., lac, tac, T3, T7 promoters for E. coli) and eukaryotic (e.g., yeast alcohol dehydrogenase (ADH1), SV40, CMV) hosts are available.

In addition, the vectors (e.g., expression vectors) typically comprise a selectable marker for selection of host cells carrying the vector, and, in the case of a replicable vector, an origin of replication. Genes encoding products which confer antibiotic or drug resistance are common selectable markers and may be used in prokaryotic (e.g., δ-lactamase gene (ampicillin resistance), Tet gene for tetracycline resistance) and eukaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated.

The invention also relates to isolated nucleic acid molecules that encode the humanized immunoglobulin or humanized light chain of the invention. In some embodiments, the invention is an isolated nucleic acid molecule encoding a humanized immunoglobulin heavy chain and a humanized immunoglobulin light chain that associate together to form a humanized immunoglobulin that has binding specificity for α4β7 integrin. Preferably, the mature humanized heavy chain is encoded by a nucleic acid comprising the nucleotide sequence of nucleotides 87-1433 of SEQ ID NO:1, and the mature humanized light chain is encoded by a nucleic acid comprising the nucleotide sequence of nucleotides 81-737 of SEQ ID NO:3.

In other embodiments, the isolated nucleic acid molecules encode a humanized immunoglobulin heavy chain and a human immunoglobulin light chain which each also comprise a signal peptide. In a specific embodiment, the isolated nucleic acid molecules encode a humanized immunoglobulin heavy chain that comprises the amino acid sequence of SEQ ID NO:2 and the humanized immunoglobulin light chain that comprises the amino acid sequence of SEQ ID NO:4. More specifically, the isolated nucleic acid molecules encode a humanized immunoglobulin heavy chain that comprises the nucleic acid sequence of SEQ ID NO:1 and the humanized immunoglobulin light chain comprises the nucleic acid sequence of SEQ ID NO:3.

In another embodiment, the invention is an isolated nucleic acid molecule encoding a humanized immunoglobulin that has binding specificity for α4β7 integrin, wherein the isolated nucleic acid comprises a first nucleotide sequence that encodes a humanized heavy chain and a second nucleotide sequence that encodes a humanized light chain. Specifically, the first nucleotide sequence comprises the nucleotide sequence of nucleotides 87-1433 of SEQ ID NO:1 and the second nucleotide sequence comprises the nucleotide sequence of nucleotides 81-737 of SEQ ID NO:3.

In another embodiment, the invention is an isolated nucleic acid molecule that encodes a humanized immunoglobulin light chain. In one embodiment, the isolated nucleic acid molecule encodes a humanized immunoglobulin light chain that is encoded by a nucleic acid comprising the nucleotide sequence of nucleotides 81-737 of SEQ ID NO:3. In some embodiments, the isolated nucleic acid molecule encodes a humanized immunoglobulin light chain that comprises a signal peptide. For example, the isolated nucleic acid molecule of the invention can encode a humanized immunoglobulin light chain that comprises the amino acid sequence of SEQ ID NO:4. For example, the isolated nucleic acid molecule can comprise the nucleotide sequence of SEQ ID NO:3.

The invention also relates to recombinant vectors (e.g., expression vectors, such as mammalian cell expression vectors, CHO expression vectors, (e.g., pLKTOK38D) that comprise a nucleic acid encoding the humanized immunoglobulin (humanized light chain and humanized heavy chain) or the humanized light chain of the invention. In one embodiment, the recombinant vector optionally further comprises a nucleotide sequence encoding a signal peptide. In a more specific embodiment, the humanized immunoglobulin or the humanized light chain of the invention is encoded in a CHO expression vector, such as pLKTOK38D.

In one embodiment, the recombinant vector comprises the nucleotide sequence of nucleotides 81-737 of SEQ ID NO:3, or comprises the nucleotide sequence of SEQ ID NO:3.

In some embodiments, the recombinant vector that comprises a nucleic acid encoding a humanized immunoglobulin light chain (e.g., a humanized immunoglobulin light chain that comprises the amino acid sequence of amino acid residues 20-238 of SEQ ID NO:4) further comprises a second nucleic acid encoding a humanized heavy chain comprising the amino acid sequence of amino acid residues 20-470 of SEQ ID NO:2. In such embodiments, the encoded heavy chain and light chain can associate together to form a humanized immunoglobulin that has binding specificity for α4β7 integrin. Optionally, the humanized heavy chain encoded by the second nucleic acid can further comprise a signal peptide. For example, the second nucleic acid can comprise the nucleotide sequence of nucleotides 87-1433 of SEQ ID NO: 1, or comprises the nucleotide sequence of SEQ ID NO:1.

In other embodiments, the recombinant vector comprises a nucleic acid encoding a humanized immunoglobulin that has binding specificity for α4β7 integrin, wherein the isolated nucleic acid comprises a first nucleotide sequence that encodes a humanized heavy chain and a second nucleotide sequence that encodes a humanized light chain. Preferably, the first nucleotide sequence comprises the nucleotide sequence of nucleotides 87-1433 of SEQ ID NO:1, and the second nucleotide sequence comprises the nucleotide sequence of nucleotides 81-737 of SEQ ID NO:3. In certain embodiments, the first nucleotide sequence and the second nucleotide sequence each further comprise a nucleotide sequence that encodes a signal peptide. For example, the isolated nucleic acid molecule can comprise the nucleotide sequence of SEQ ID NO:1, and the nucleotide sequence of SEQ ID NO:3.

Method of Producing Humanized Immunoglobulins Having Specificity for α4β7 Integrin

Another aspect of the invention relates to a method of preparing a humanized immunoglobulin which has binding specificity for α4β7 integrin. The humanized immunoglobulin can be produced, for example, by the expression of one or more recombinant nucleic acids encoding a humanized immunoglobulin having binding specificity for α4β7 integrin in a suitable host cell.

Host cells which produce a humanized immunoglobulin having binding specificity for α4β7 integrin can be produced using any suitable method. For example, the expression constructs (e.g., a mammalian cell expression vector) described herein can be introduced into a suitable host cell, and the resulting cell can be maintained (e.g., in culture, in an animal, in a plant) under conditions suitable for expression of the construct(s) or vector(s). Suitable host cells can be prokaryotic, including bacterial cells such as E. coli (e.g., strain DH5α™ (Invitrogen, Carlsbad, Calif.), B. subtilis and/or other suitable bacteria; eukaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eukaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096, CHO DG44 (Urlaub, G. and Chasin, L A., Proc. Natl. Acad. Sci. USA, 77(7):4216-4220 (1980))), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NS0 cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).) In some embodiments, the host cell is an isolated host cell and is not part of a multicellular organism (e.g., plant or animal). In preferred embodiments, the host cell is a non-human host cell.

The present invention also relates to cells comprising a vector of the invention (e.g., an expression vector). For example, a nucleic acid (i.e., one or more nucleic acids) encoding the heavy and light chains of a humanized immunoglobulin having binding specificity for α4β7 integrin, or a construct (i.e., one or more constructs) comprising such nucleic acid(s), can be introduced into a suitable host cell by a method appropriate to the host cell selected (e.g., transformation, transfection, electroporation, infection), such that the nucleic acid(s) are operably linked to one or more expression control elements (e.g., in a vector, in a construct created by processes in the cell, integrated into the host cell genome). Host cells can be maintained under conditions suitable for expression (e.g., in the presence of inducer, suitable media supplemented with appropriate salts, growth factors, antibiotic, nutritional supplements, etc.), whereby the encoded polypeptide(s) are produced. If desired, the encoded protein (e.g., humanized immunoglobulin) can be isolated, for example, from the host cells, culture medium, or milk. This process encompasses expression in a host cell of a transgenic animal or plant (tobacco) (see e.g., WO 92/03918).

Fusion proteins can be produced in which a humanized immunoglobulin or immunoglobulin chain is linked to a non-immunoglobulin moiety (i.e., a moiety which does not occur in immunoglobulins as found in nature) in an N-terminal location, C-terminal location or internal to the fusion protein. For example, some embodiments can be produced by the insertion of a nucleic acid encoding immunoglobulin sequences into a suitable expression vector, such as a pET vector (e.g., pET-15b, Novagen), a phage vector (e.g., pCANTAB 5 E, Pharmacia), or other vector (e.g., pRIT2T Protein A fusion vector, Pharmacia). The resulting construct can be introduced into a suitable host cell for expression. Upon expression, some fusion proteins can be isolated or purified from a cell lysate by means of a suitable affinity matrix (see, e.g., Current Protocols in Molecular Biology (Ausubel, F. M. et al., Eds., Vol. 2, Suppl. 26, pp. 16.4.1-16.7.8 (1991)).

The invention relates to a host cell that comprises a recombinant nucleic acid encoding the humanized immunoglobulin (humanized light chain and humanized heavy chain) or the humanized light chain of the invention. For example, in some embodiments, the host cell comprises a recombinant vector (e.g., expression vector, mammalian expression vector, CHO expression vector, pLKTOK38D expression vector) of the invention as referred to herein. In a specific embodiment, the host cell is a CHO cell, such as CHO DG44.

The invention also relates to a method of preparing a humanized immunoglobulin comprising maintaining a host cell of the invention as described herein (e.g., a host cell that contains one or more recombinant nucleic acids that encode a humanized immunoglobulin (e.g., a humanized light chain and a humanized heavy chain, a humanized light chain only) of the invention) under conditions appropriate for expression of a humanized immunoglobulin. For expression of a humanized immunoglobulin or light chain of a humanized immunoglobulin, a host cell can be maintained under any suitable conditions. For example a host cell can be cultured on a substrate or in suspension. In one embodiment, the host cells are maintained under appropriate conditions, humanized immunoglobulin chains are expressed and a humanized immunoglobulin is produced. In some embodiments, the method further comprises the step of isolating the humanized immunoglobulin.

Therapeutic Methods and Compositions

The present invention provides humanized immunoglobulins which (1) can bind α4β7 integrin in vitro and/or in vivo; and/or (2) can modulate an activity or function of an α4β7 integrin, such as (a) binding function (e.g., the ability of α4β7 integrin to bind to MAdCAM-1, fibronectin and/or VCAM-1) and/or (b) leukocyte infiltration function, including recruitment and/or accumulation of leukocytes in tissues (e.g., the ability to inhibit lymphocyte migration to intestinal mucosal tissue). Preferably, the humanized immunoglobulins are capable of selectively binding α4β7 in vitro and/or in vivo, and inhibiting α4β7 mediated interactions. In one embodiment, a humanized immunoglobulin can bind an α4β7 integrin, and can inhibit binding of the α4β7 integrin to one or more of its ligands (e.g., MAdCAM-1, VCAM-1, fibronectin), thereby inhibiting leukocyte infiltration of tissues (including recruitment and/or accumulation of leukocytes in tissues), preferably selectively. Such humanized immunoglobulins can inhibit cellular adhesion of cells bearing an α4β7 integrin to vascular endothelial cells in mucosal tissues, including gut-associated tissues, lymphoid organs or leukocytes (especially lymphocytes such as T or B cells) in vitro and/or in vivo. In a particularly preferred embodiment, a humanized immunoglobulin can inhibit the interaction of α4β7 with MAdCAM-1 and/or fibronectin.

The humanized immunoglobulins of the present invention are useful in a variety of processes with applications in research, diagnosis and therapy. For instance, they can be used to detect, isolate, and/or purify α4β7 integrin or variants thereof (e.g., by affinity purification or other suitable methods), and to study α4β7 integrin structure (e.g., conformation) and function.

The humanized immunoglobulins of the present invention can also be used in diagnostic applications (e.g., in vitro, ex vivo) or to modulate α4β7 integrin function in therapeutic (including prophylactic) applications.

For example, the humanized immunoglobulins of the present invention can be used to detect and/or measure the level of an α4β7 integrin in a sample (e.g., tissues or body fluids, such as an inflammatory exudate, blood, serum, bowel fluid, on cells bearing an α4β7 integrin). For example, a sample (e.g., tissue and/or body fluid) can be obtained from an individual and a suitable immunological method can be used to detect and/or measure α4β7 integrin expression, including methods such as enzyme-linked immunosorbent assays (ELISA), including chemiluminescence assays, radioimmunoassay, and immunohistology.

In one embodiment, a method of detecting a selected α4β7 integrin in a sample is provided, comprising contacting a sample with a humanized immunoglobulin of the present invention under conditions suitable for specific binding of the humanized immunoglobulin to the α4β7 integrin and detecting antibody-α4β7 integrin complexes which are formed. In an application of the method, humanized immunoglobulins can be used to analyze normal versus inflamed tissues (e.g., from a human) for α4β7 integrin reactivity and/or expression (e.g., immunohistologically)) to detect associations between inflammatory bowel disease (IBD) or other conditions and increased expression of α4β7 (e.g., in affected tissues).

The humanized immunoglobulins of the present invention permit immunological methods of assessment of the presence of α4β7 integrin in normal versus inflamed tissues, through which the presence of disease, disease progress and/or the efficacy of anti-α4β7 integrin therapy in inflammatory disease can be assessed. The humanized immunoglobulins of the present invention can also be used to modulate (e.g., inhibit (reduce or prevent)) binding function and/or leukocyte (e.g., lymphocyte, monocyte) infiltration function of α4β7 integrin. For example, humanized immunoglobulins which inhibit the binding of α4β7 integrin to a ligand (i.e., one or more ligands) can be administered according to the method in the treatment of diseases associated with leukocyte (e.g., lymphocyte, monocyte) infiltration of tissues (including recruitment and/or accumulation of leukocytes in tissues), particularly of tissues which express the molecule MAdCAM-1.

An effective amount of a humanized immunoglobulin of the present invention (i.e., one or more) is administered to an individual (e.g., a mammal, such as a human or other primate) in order to treat such a disease. For example, inflammatory diseases, including diseases which are associated with leukocyte infiltration of the gastrointestinal tract (including gut-associated endothelium), other mucosal tissues, or tissues expressing the molecule MAdCAM-1 (e.g., gut-associated tissues, such as venules of the lamina propria of the small and large intestine; and mammary gland (e.g., lactating mammary gland)), can be treated according to the present method. Similarly, an individual having a disease associated with leukocyte infiltration of tissues as a result of binding of leukocytes to cells (e.g., endothelial cells) expressing MAdCAM-1 can be treated according to the present invention.

In a particularly preferred embodiment, diseases which can be treated accordingly include inflammatory bowel disease (IBD), such as ulcerative colitis, Crohn's disease, ileitis, Celiac disease, nontropical Sprue, enteropathy associated with seronegative arthropathies, microscopic or collagenous colitis, eosinophilic gastroenteritis, or pouchitis resulting after proctocolectomy, and ileoanal anastomosis.

Pancreatitis and insulin-dependent diabetes mellitus are other diseases which can be treated using the present method. It has been reported that MAdCAM-1 is expressed by some vessels in the exocrine pancreas from NOD (nonobese diabetic) mice, as well as from BALB/c and SJL mice. Expression of MAdCAM-1 was reportedly induced on endothelium in inflamed islets of the pancreas of the NOD mouse, and MAdCAM-1 was the predominant addressin expressed by NOD islet endothelium at early stages of insulitis (Hanninen, A., et al., J. Clin. Invest., 92: 2509-2515 (1993)). Further, accumulation of lymphocytes expressing α4β7 within islets was observed, and MAdCAM-1 was implicated in the binding of lymphoma cells via α4β7 to vessels from inflamed islets (Hanninen, A., et al., J. Clin. Invest., 92: 2509-2515 (1993)).

Examples of inflammatory diseases associated with mucosal tissues which can be treated according to the present method include mastitis (mammary gland), cholecystitis, cholangitis or pericholangitis (bile duct and surrounding tissue of the liver), chronic bronchitis, chronic sinusitis, asthma, and graft versus host disease (e.g., in the gastrointestinal tract). As seen in Crohn's disease, inflammation often extends beyond the mucosal surface, accordingly chronic inflammatory diseases of the lung which result in interstitial fibrosis, such as hypersensitivity pneumonitis, collagen diseases, sarcoidosis, and other idiopathic conditions can be amenable to treatment.

The humanized immunoglobulin is administered in an effective amount which inhibits binding of α4β7 integrin to a ligand thereof. For therapy, an effective amount will be sufficient to achieve the desired therapeutic (including prophylactic) effect (such as an amount sufficient to reduce or prevent α4β7 integrin-mediated binding and/or signaling, thereby inhibiting leukocyte adhesion and infiltration and/or associated cellular responses). The humanized immunoglobulin can be administered in a unit dose or multiple doses. The dosage can be determined by methods known in the art and can be dependent, for example, upon the individual's age, sensitivity, tolerance and overall well-being. Suitable dosages for antibodies can be from about 0.1 mg/kg body weight to about 10.0 mg/kg body weight per treatment. In particular embodiments, the dose administered is about 0.3 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg.

According to the method, the humanized immunoglobulin can be administered to an individual (e.g., a human) alone or in conjunction with another agent. A humanized immunoglobulin can be administered before, along with or subsequent to administration of the additional agent. In one embodiment, more than one humanized immunoglobulin which inhibits the binding of α4β7 integrin to its ligands is administered. In another embodiment, a monoclonal antibody, such as an anti-MAdCAM-1, anti-VCAM-1, or anti-ICAM-1 antibody, which inhibits the binding of leukocytes to an endothelial ligand is administered in addition to a humanized immunoglobulin of the present invention. In yet another embodiment, an additional active ingredient (e.g., an antiinflammatory compound, such as sulfasalazine, another non-steroidal antiinflammatory compound, or a steroidal antiinflammatory compound) can be administered in conjunction with a humanized immunoglobulin of the present invention.

A variety of routes of administration can be used, including, but not necessarily limited to, parenteral (e.g., intravenous, intraarterial, intramuscular, subcutaneous injection), oral (e.g., dietary), topical, inhalation (e.g., intrabronchial, intranasal or oral inhalation, intranasal drops), or rectal, depending on the disease or condition to be treated. Parenteral administration is a preferred mode of administration.

Formulation will vary according to the route of administration selected (e.g., solution, emulsion). An appropriate composition comprising the humanized antibody to be administered can be prepared in a physiologically acceptable vehicle or carrier. The composition can comprise multiple doses or be a unit dose composition. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See, generally, Remington's Pharmaceutical Sciences, 17th Edition, Mack Publishing Co., PA, 1985). For inhalation, the compound can be solubilized and loaded into a suitable dispenser for administration (e.g., an atomizer, nebulizer or pressurized aerosol dispenser).

The invention also relates to a method of inhibiting the interaction of a first cell bearing α4β7 with a second cell bearing a ligand thereof. In one embodiment, the method comprises contacting the first cell with an effective amount of a humanized immunoglobulin or humanized light chain of the invention.

The invention also relates to a method of inhibiting leukocyte infiltration of mucosal tissue. In one embodiment, the method comprises administering an effective amount of a humanized immunoglobulin or humanized light chain of the invention to a patient.

The invention also relates to a method for treating cancer (e.g., an α4β7 positive tumor, such as a lymphoma). In one embodiment, the method comprises administering an effective amount of a humanized immunoglobulin or humanized light chain or immunoconjugate of the invention to a patient.

The invention also relates to a method for treating a disease associated with leukocyte infiltration of tissues expressing the molecule MAdCAM-1. The method comprises administering to a patient in need thereof an effective amount of a humanized immunoglobulin or humanized light chain of the invention. In particular embodiments, the disease is mastitis, chronic sinusitis or graft versus host disease. In some embodiments, the disease is a disease associated with leukocyte infiltration of tissues as a result of binding of leukocytes expressing α4β7 integrin to gut-associated endothelium expressing the molecule MAdCAM-1. In particular embodiments, the disease is gastritis (e.g., eosinophilic gastritis), pancreatitis, insulin-dependent diabetes mellitus, cholecystitis, cholangitis, pericholangitis.

The invention also relates to a method for treating inflammatory bowel disease in a patient. In one embodiment, the method comprises administering to the patient an effective amount of a humanized immunoglobulin or humanized light chain of the invention. In some embodiments, the inflammatory bowel disease is ulcerative colitis or Crohn's disease. In other embodiments, the inflammatory bowel disease is Celiac disease, enteropathy associated with seronegative arthropathies, microscopic or collagenous colitis, gastroenteritis (e.g., eosinophilic gastroenteritis), or pouchitis.

The entire teachings of all documents cited herein are hereby incorporated herein by reference.

Exemplification

The present invention will now be illustrated by the following Examples, which are not intended to be limiting in any way.

### EXAMPLE 1

### Humanized Act-1 Antibody that Binds α4β7

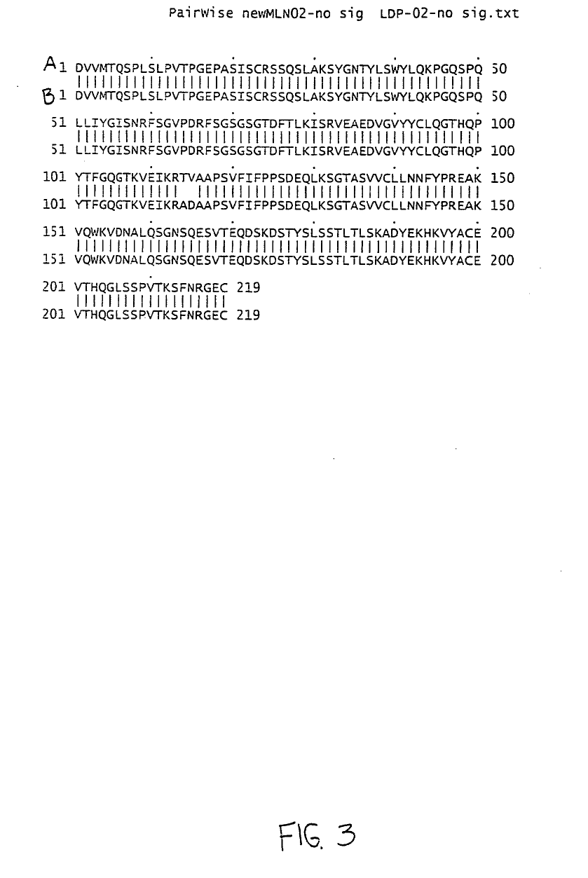

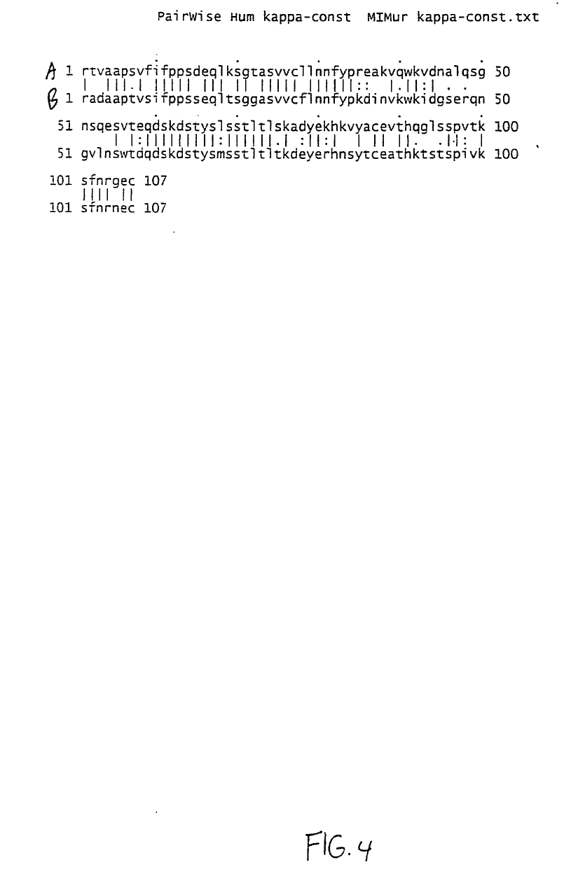

A humanized immunoglobulin (referred to herein as MLN02) that comprises the CDRs of the murine Act-1 antibody and binds α4β7 integrin was produced. The heavy chain of the humanized immunoglobulin comprises mutations in the Fc portion to reduce the ability to fix complement (SEQ ID NO:2). The amino acid sequence of the mature light chain of the humanized immunoglobulin (amino acid residues 20-238 of SEQ ID NO:4) is presented in FIG. 3. As shown in FIG. 3, the amino acid sequence of the humanized light chain of MLN02 is different from the amino acid sequence of the humanized light chain of another humanized antibody that contains the CDRs of the murine Act-1 antibody (referred to herein as LDP-02, see, WO 98/06248). In particular the amino acid sequences of MLN02 and LDP-02 differ at positions 114 and 115 of the mature proteins (amino acid residues 133 and 134 of SEQ ID NO:4, and amino acid residues 114 and 115 of SEQ ID NO:5, respectively). The positions of the amino acid differences between MLN02 and LDP-02 correspond to amino acid positions 2 and 3 of the constant region of kappa light chain. (FIG. 4)

As shown in the alignment of the constant regions of human (Homo sapiens) kappa light chain (SEQ ID NO:6) and mouse (Mus musculus) kappa light chain (SEQ ID NO:7), the amino acid residues Thr and Val (which are present in MLN02) are present in the constant region of the human kappa light chain, whereas the amino acid residues Ala and Asp (which are present in LDP-02) are present in the constant region of the mouse kappa light chain (FIG. 4). Accordingly, the humanized light chain of MLN02 (e.g., amino acid residues 20-238 of SEQ ID NO:4) is more human than the light chain of LDP-02.

The degree to which a humanized immunoglobulin resembles a natural human immunoglobulin is associated with the magnitude of immune responses that are directed against the humanized immunoglobulin (e.g., human anti-humanized antibody (HAHA) response) when it is administered to a human. A HAHA response can limit, reduce or eliminate the efficacy of a humanized immunoglobulin. The humanized immunoglobulin MLN02 is more human than LDP-02 and therefore can be administered to humans with lower risk of producing a HAHA response that can reduce or eliminate efficacy of the humanized immunoglobulin.

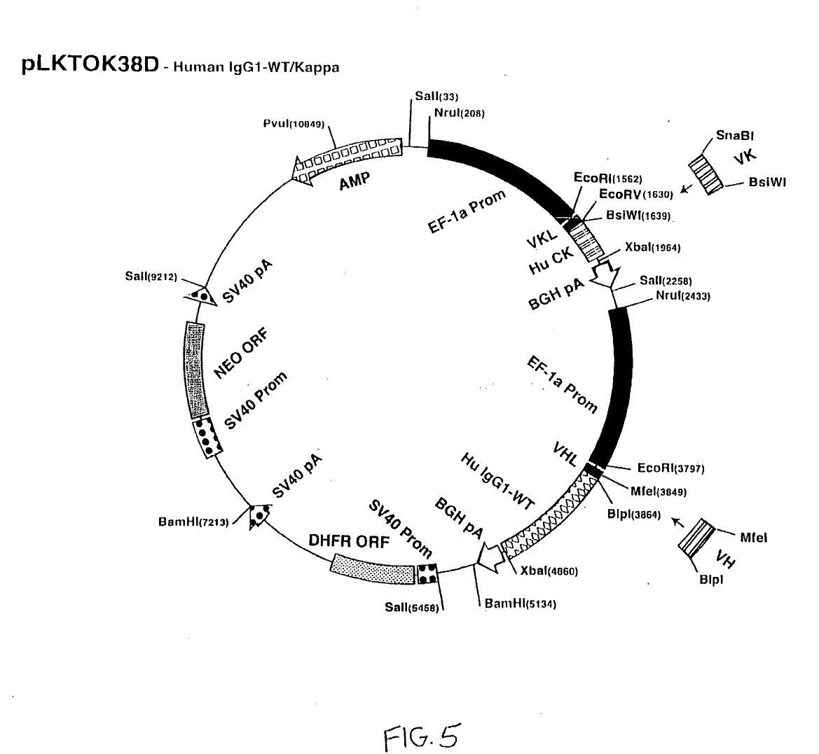

An E. coli strain comprising the pLKTOK38D vector (also referred to as pTOK38MLN02-TV) (FIG. 5), which encodes the humanized antibody MLN02 (encodes the humanized heavy chain and humanized light chain of MLN02) and is suitable for expressing MLN02 in CHO cells, was deposited under the provisions of the Budapest Treaty on Nov. 10, 2005, on behalf of Millennium Pharmaceuticals, Inc., 75 Sidney Street, Cambridge, Mass. 02219, U.S.A., at the American Type Culture Collection, 10801 University Boulevard, Manassas, Va. 20110-2209, U.S.A., under Accession No. PTA-7213.

### EXAMPLE 2

### Binding Data

The ability of MLN02 to inhibit binding of α4β7 to human soluble MAdCAM-1 was assessed using AlamarBlue® (cell growth and cytotoxicity indicator dye, Trek Diagnostic Systems), α4β7 integrin-expressing RPMI-8866 cells (a human B cell lymphoma), and a MAdCAM-1 chimera comprising the entire extracellular domain of human MAdCAM-1 fused to the Fc region of a human IgG₁ (i.e., CH₂ and CH₃ of human IgG₁).

The MAdCAM-Fc was diluted to 2 μg/ml in PBS, and 100 μl of the solution were added to wells of an 96 well assay plate. The plate was sealed and kept at 4° C. for 1-3 days. On the assay day, the MAdCAM-Fc coating solution was poured out of the wells of the plate, and 150 μl of blocking buffer was added to each well. The plate was then kept at 37° C. in a CO₂ oven for 1 hour. Then, the blocking solution was poured out of the wells and the plates were blotted dry on a paper towel. 50 μl of serially diluted antibody to be tested (e.g., MLN02, LDP-02) were added to the inner 60 wells of assay plate, and then 50 μl of RPMI-8866 cells (at 4×10⁶ cells/ml) were added to each well. The plate was covered with the plate lid and gently agitated on an orbital shaker for 1-2 minutes. Then, the plate was incubated at 37° C. in a CO₂ oven for 1 hour. Then, the solution was removed from the wells using a hand-held vacuum aspirator. The wells were washed by adding 200 μl PBS to the side of each well (without disturbing the cell layer at the bottom of the well), and removing unbound cells by aspiration. The plate was rotated 180° and washed once more. 20% Alamar Blue was added to each well, the plate was covered with the plate lid and returned to the 37° C. CO₂ oven for 3 hours. Then the plates were read at 530 nm/590 nm on a fluorescence plate reader to obtain Relative Fluorescence Units (RFU) which are proportional the amount of cells that adhered to MAdCAM-Fc that was immobilized to the wells.

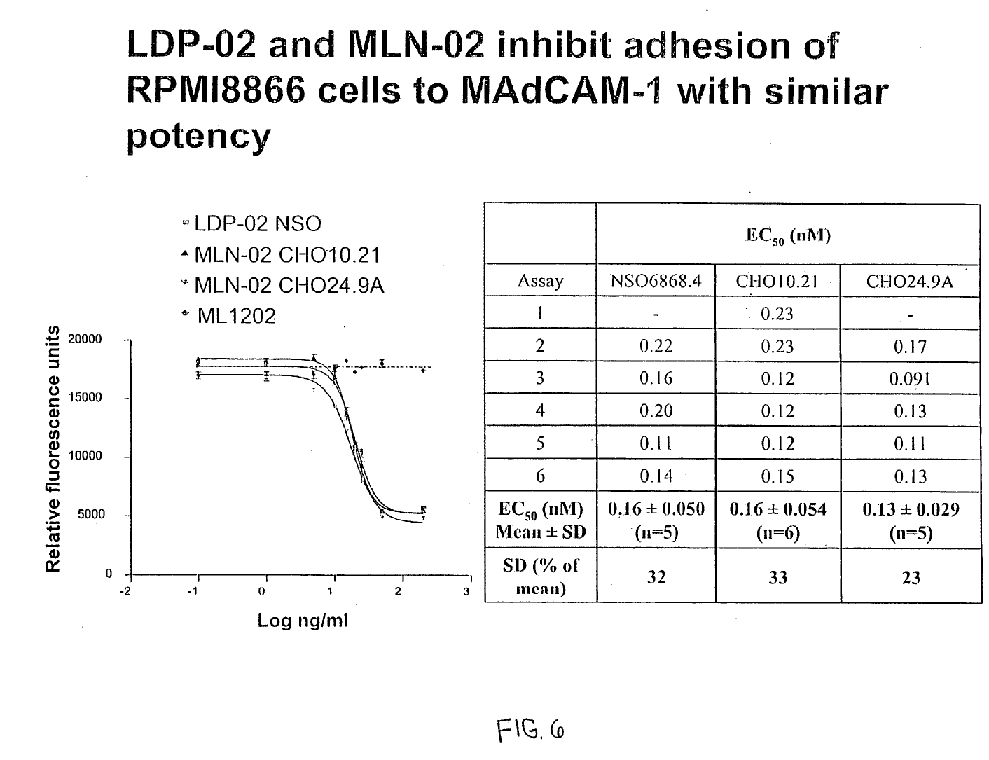

The results show that LDP-02 that was produced in NS0 cells (LDP-02 NS0) and MLN02 that was produced by two different clones of a CHO cell line (MLN02 CHO10.21 and MLN02CHO24.9A) inhibited adhesion of RPMI-8866 cells to immobilized MAdCAM-1 with similar potency (FIG. 6). A control antibody (ML 1202) that does not bind α4β7 did not inhibit adhesion of RPMI-8866 cells in the assay.

LDP-02 binds α4β7 with a higher affinity than the ACT-1 antibody (see, Example 4B in WO 98/06248). Therefore, the results of the binding assay which demonstrate the MLN02 binds α4β7 with substantially the same potency as LDP-02, indicates that MLN02 also binds α4β7 with higher affinity than the ACT-1 antibody.

### EXAMPLE 3

### Biochemical and Biophysical Properties of MLN02

Biochemical and biophysical characterization of MLN02 was performed using MLN02 produced in CHO cells that contained an expression vector that encodes the humanized light chain and humanized heavy chain of MLN02 (pLKTOK38D). Several samples of MLN02 produced by difference CHO cell clones (samples 32A, 10-27A, 10-21, 8-18A, 10-27-4, 24-9A) were produced. In addition several reference standards ware prepared.

A LDP-02 reference standard (that contained LDP-02 produced using NS0 cells) was formulated in 20 mM sodium citrate, pH 6.0, containing 125 mM sodium chloride, the final concentration of LDP-02 was 4.6 mg/mL.

A LDP-02 reference standard (that contained LDP-02 produced using CHO cells) was formulated in 90 mM phosphate, 200 mM arginine and 0.02% Tween-20 (pH 6.3), the final concentration of LDP-02 was 56.8 mg/mL. This reference standard is referred to as ADB-MLN02-04-002.

A LDP-02 reference standard (that contained LDP-02 produced using CHO cells) was formulated in 20 mM sodium citrate, pH 6.0 containing 125 mM sodium chloride. This reference standard is referred to as 2353.

1. Size-Exclusion Chromatography (SEC)

Test samples were analyzed by SEC using a Tosoh Biosep G3000 SWXL column on a Waters HPLC system. This method allowed for the separation of antibody monomer from aggregates, degradation products, and other product- and nonproduct-related species. Briefly, 100 μg of the test sample was injected onto the column and eluted with an isocratic phosphate-sodium chloride buffer system at pH 6.8 using a flow rate of 0.5 mL/min. Elution of the protein species was monitored at 280 nm, and the total peak area was assessed to determine purity.

Analysis of the sample proteins indicated that there was little difference in the percentage of monomer measured in any of the samples (Table 1). Table 1 presents the results obtain in 2 independent assays. Standards (reference samples) were included in each assay and comparisons of retention times between assays should not be made. However, the differences in retention time between reference samples and MLN02 samples were consistent in each assay. The percentage of intact monomer varied from 98.55-99.64% in the CHO-produced proteins, corresponding to 99.59% in the reference standard ADB-MLN02-04-002. One difference, which was noted, was that the retention of the intact monomer produced by several CHO clones was consistently 0.07-0.08 minutes slower than the reference LDP-02 sample produced in NS0 cells or either of the LDP-02 reference standards produced using CHO cells. This difference has been attributed to the difference in the amino acid sequences of MLN02 and LDP-02 described above, and suggests that MLN02 is more compact that LDP-02.

TABLE 1

| | % | | | Retention |
|---|---|---|---|---|
| | HMW | % | % LMW | Time, |
| SAMPLE | Species | Monomer | Species | monomer (min) |
| Reference Standard #1 | 0.36 | 99.64 | ND | 17.44 |
| ADB-MLN02-04-002^{a} | | | | |
| 2353^{a} | 0.85 | 98.80 | 0.35 | 17.42 |
| 32A^{a} | 0.98 | 99.02 | ND | 17.51 |
| 10-27A^{a} | 0.92 | 99.03 | 0.05 | 17.51 |
| Reference Standard #2 | 0.47 | 99.53 | ND | 17.25 |
| ADB-MLN02-04-002^{b} | | | | |
| 10-21^{b} | 0.88 | 99.12 | ND | 17.33 |
| 8-18A^{b} | 0.36 | 99.64 | ND | 17.33 |
| 10-27-4^{b} | 0.39 | 99.64 | ND | 17.33 |
| 24-9A^{b} | 1.45 | 98.55 | ND | 17.32 |

^{a}= SEC run #1
^{b= SEC run #2}
ND = none detected

2. SDS-PAGE

In order to assist in establishing identity, characterize the first preparation, and assess purity, MLN02 test samples were subjected to sodium dodecyl sulfate polyacrylamide gel electrophoresis (SDS-PAGE) under non-reducing and reducing conditions. Under reducing conditions, test samples of MLN02 were analyzed using a 4-20% polyacrylamide Tris-glycine gel. Under non-reducing conditions, test samples of MLN02 were analyzed using a 4-12% polyacrylamide Tris-glycine gel.

All CHO cell produced MLN02 samples were analyzed by SDS-PAGE under reduced and non-reduced conditions in comparison with a reference standard (ABD-MLN02-04-002). The ABD-MLN02-04-002 reference standard was previously compared to the LDP-02 reference standard and the results under reducing conditions were substantially the same. Under non-reducing conditions, a minor species (<3% of total) was identified in ABD-MLN02-04-002 but was absent in the LDP-02 reference standard.

Under reducing conditions, MLN02 produced by CHO clones separated into two major band areas at approximately 50,000 Daltons and approximately 28,000 Daltons, corresponding to the heavy- and light-chain. The measured intensity of the heavy and light chains for MLN02 produced by CHO clones was found to be between 85.2% and 96.4% of the total band density, in comparison with independent measurements of 91.2% and 96.4% for the reference standard (ABD-MLN02-04-002). Although additional minor bands were present in several of the samples, their relative percentages were less than 2% in all cases.

Under non-reducing conditions, one major and one minor band corresponding to intact IgG was present in MLN02, with molecular weights of approximately 172,000 Daltons and 154,000 Daltons. The measured intensity of the intact IgG for the reference standard (ABD-MLN02-04-002) was 78.1% and 85.5% in two independent measurements. The measured values for MLN02 produced by the CHO cell clones were 79.8-84.1%. Minor amounts of a higher-molecular weight aggregate were detected in samples produced by CHO cell clones 10-27A, 8-18A, 10-21, 10-27-4 and 24-9A, but other species were minor and comparable amounts were detected in the reference standard (ABD-MLN02-04-002) and the MLN02 produced by the CHO clones.

3. IsoElectric Focusing

Test samples of the invention were analyzed by Isoelectric Focusing (IEF) using an agarose gel with a pH gradient of 6.0-10.5. The results obtained were compared to the IEF standards.

The overall pI range of MLN02 produced by CHO cell clones was found to be 7.66-8.00, with only small variations in the individual samples. In comparison, a more acidic range of 7.62-7.85 for the LDP-02 reference standard produced by CHO cells (ADB-MLN02-04-002), and 7.59-7.89 for the LDP-02 reference standard produced by clone 2353, was observed.

The pI profiles for the LDP-02 samples produced in CHO cells were found to be similar, with only small differences in individual species distribution. Comparison of CHO cell produced standard ADB-MLN02-04-002 and the LDP-02 standard produced in NS0 cells revealed a larger variation in species distribution, although the overall pI range was found to be similar. The minor differences observed may be due to the ages of the ADB-MLN02-04-002 standard and the LDP-02 standard produced in NS0, as deamidation and other degradation processes are known to alter pI values.

The predicted pI for LDP-02 produced in NS0 cells was 7.9, and the predicted pI for MLN02 was 8.09 based upon the primary amino acid sequence. The pI shift observed in MLN02 relative to LDP-02 produced using NS0 cells is consistent with the different amino acid sequences of the light chains of the humanized antibodies, specifically the V at position 134 of MLN02.

4. Glycosolation Pattern

The carbohydrate profile of the LDP-02 reference standards and MLN02 samples were generated by the separation of free fluorescently-labeled carbohydrates by reverse-phase HPLC. Briefly, intact glycans were released from the protein samples of the invention (100 μg) by overnight digestion with PNGaseF, exchanged into an appropriate buffer system, and then labeled with 2-aminobenzamide (2-AB) on their reducing ends. The labeled glycans were fractionated on a Waters HPLC system using a Prozyme GlykoSep N column with an acetonitrile/ammonium formate buffer system. They were then detected by fluorescence emission at 420 nm. Individual carbohydrate moieties were tentatively identified by comparing retention times to historical results obtained from carbohydrate standards, as well as from a well-characterized humanized antibody that does not bind α4β7.

Carbohydrate profiling of the LDP-02 and MLN02 samples produced by CHO clones showed some distinct changes which appear to be attributable to the manufacturing cell line (Table 2). The decreased levels of the more complex glycoforms (GIF, G2F, G2S1) on these samples compared to NS0-expressed LDP-02, was of particular interest. The sum of these three species in the NS0-expressed LDP-02 protein was approximately 73%.

The LDP-02 or MLN02 proteins produced using CHO cells were mainly glycosylated with G0F oligosaccharide (agalactosylated, core fucosylated biantennary complex type), accounting for 58% to 76% of the total glycoforms observed. The GIF species (monogalactosylated core fucosylated biantennary complex type) accounted for only 15-30% of the total glycans, and the G2F (digalactosylated core fucosylated biantennary complex type) species accounted for less than 5% in all samples tested. Minor amounts of additional unidentified species are present.

Although cell culture conditions may have played a small role in the difference observed in the MLN02 glycoforms, the cell type influenced the carbohydrate profile more significantly.

TABLE 2

| | | | | | TBD |
|---|---|---|---|---|---|
| | G0F | G1F | G2F | G2S1 | (late eluting) |
| LDP-02 produced | 14% | 43% | 23% | 7% | 7% |
| using NS0 cells | | | | | |
| ADB-MLN02-04-002 | 75% | 20% | 2% | <1% | ND |
| 2353 | 66% | 26% | 2% | <1% | ND |
| 32A | 71% | 19% | 2% | <1% | ND |
| 10-270A | 58% | 30% | 4% | <1% | ND |
| 8-18A | 71% | 23% | 2% | <1% | ND |
| 10-21 | 71% | 22% | 2% | ND | ND |
| 10-27-4 | 76% | 15% | 1% | ND | ND |
| 24-9A | 73% | 19% | 1% | ND | ND |

ND = not detected

5. Peptide Mapping

LDP-02 standards and MLN02 samples were reduced at pH 8.7 under denaturing conditions and then treated with iodoacetic acid to block free thiol groups. Samples were then desalted and digested with trypsin, and the resultant peptides were separated on a C18 column using a water/acetonitrile/TFA gradient with detection at 215 nm. The MLN02 sample profiles were compared to the LDP-02 reference standard by identifying the retention time of major peaks in both samples.

The results of the peptide mapping of MLN02 produced by CHO cell clones indicated that the proteins were mostly comparable to the LDP-02 reference standard produced in NS0 cells. However, a few peaks were identified on the map which clearly differed from the LDP-02 reference. In particular, differences in the profiles were noted for some or all of the MLN02 samples produced by CHO cell clones at approximately 39, 58, 95, and 105 minutes on the map. The most notable change was the appearance of a new peak, broadly centered at approximately 105 minutes retention time present in all of the MLN02 samples produced by CHO clones, but not seen in LDP-02 reference sample 2353, which was also produced by expression in a CHO cell. This indicated that the differences seen in the MLN02 samples and LDP-02 samples were most likely due to the different amino acid sequences of the light chains of the humanized antibodies.

### EXAMPLE 4

### Pharmacokinetic/Pharmacodynamic in Cynomolgus Monkeys

This study was done to provide pharmacokinetic/pharmacodynamic (PK/PD) information to compare LDP-02 and MLN02. Briefly, two groups of four male test animals (cynomolgus monkeys, Macaca fascicularis, 3-5 kg) were administered either LDP-02 (produced using a NS0 cell line) or MLN02 (produced using a CHO cell line) at a dose of 10 mg/kg. The test material was administered to the test animals by intravenous (IV) infusion over approximately 30 minutes on Day 1, and then by slow bolus IV over approximately one minute on Days 4, 8, 11, and 15. A blood sample was collected on Day 1 predose, at the end of infusion, and then 1, 2, 4, 8, 10, 24, and 48 hours after the end of infusion. Additional blood samples were collected on Day 4, 8, and 11 before dosing and 0.5 hours postdose. On day 15 blood samples were collected before dosing, 0.5, 1, 2, 4, 8, 10, 24, 48, 72 and 168 hours after the end of infusion. The blood samples were centrifuged and serum removed in four aliquots (˜50 μL) for PK studies.

Serum concentration of LDP-02 and MLN02 were determined using a sandwich ELISA assay. A mouse antibody that binds LDP-02 and MLN02 was immobilized and used to capture LDP-02 or MLN02 from serum samples. Captured LDP-02 or MLN02 was detected using a mouse anti-human IgG antibody labeled with horse radish peroxidase.

Qualitative measures of humanized antibody bound to α4β7 on the surface of cells or of the number of α4β7 sites on the surface of cells were obtained using flow cytometry.

For measuring bound antibody, two 90 μl aliquots of a blood sample were used. The aliquots were place in separate tubes (tube 1 and tube 2) and each was washed twice using PBS/0.1% BSA. The cells in tube 1 were stained with antibodies that bind CD4, CD8 and a secondary donkey anti-human IgG antibody which binds and detects MLN02 and LDP-02. The cells in tube 2 were stained with antibodies that bind CD4, CD8 and a control IgG. The cells in each tube were incubated with the staining antibodies for 30 minutes at room temperature. Then the red blood cells in the tubes were lysed, and the white blood cells were fixed before being analyzed on a fluorescence activated flow cytometer. This assay was used to obtain qualitative measures of the amount of humanized antibody that was bound to α4β7 on the surface of CD4⁺ or CD8⁺ cells.

A similar assay was used to detect α4β7 sites that were not bound by humanized antibody. Again, two 90 ml aliquots of a blood sample were used. The aliquots were place in separate tubes (tube 1 and tube 2). The cells in tube 1 were stained with antibodies that bind CD3, CD4, CD8 and a labeled MLN02 (labeled with alexa 647). The cells in tube 2 were stained with antibodies that bind CD3, CD4, and CD8. The cells in each tube were incubated with the staining antibodies for 30 minutes at room temperature. Then the red blood cells in the tubes were lysed, and the white blood cells were fixed before being analyzed on a fluorescence activated flow cytometer. This assay was used to obtain qualitative measures of the amount of α4β7 on the surface of CD4⁺ or CD8⁺ cells that was not bound by humanized antibody.

The PK (serum concentrations of LDP-02 and MLN02) data revealed that the exposure (area under the concentration time curve; AUC) for animals administered LDP-02 and MLN02 were substantially similar, but that the AUC was slightly higher for LDP-02. (Table 3)

TABLE 3

| | | |
|---|---|---|
| | AUC_{0-72 hours} | AUC_{0-168 hours} |
| Humanized | (mg * hr/mL) | (mg * hr/mL) |

| Antibody | Mean | SD | Mean | SD |
|---|---|---|---|---|
| LDP-02 | 16800 | 3000 | 73800 | 6900 |
| MLN02 | 11700 | 2350 | 52300^{#} | 10000^{#} |

^{#}one animal developed a high titre primate anti-humanized antibody antibody response and was excluded form the analysis.

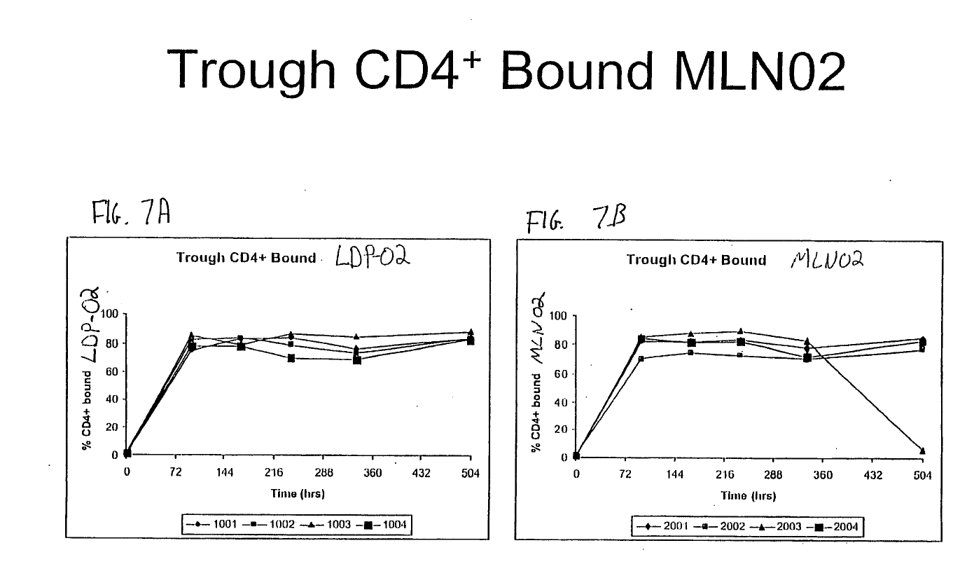

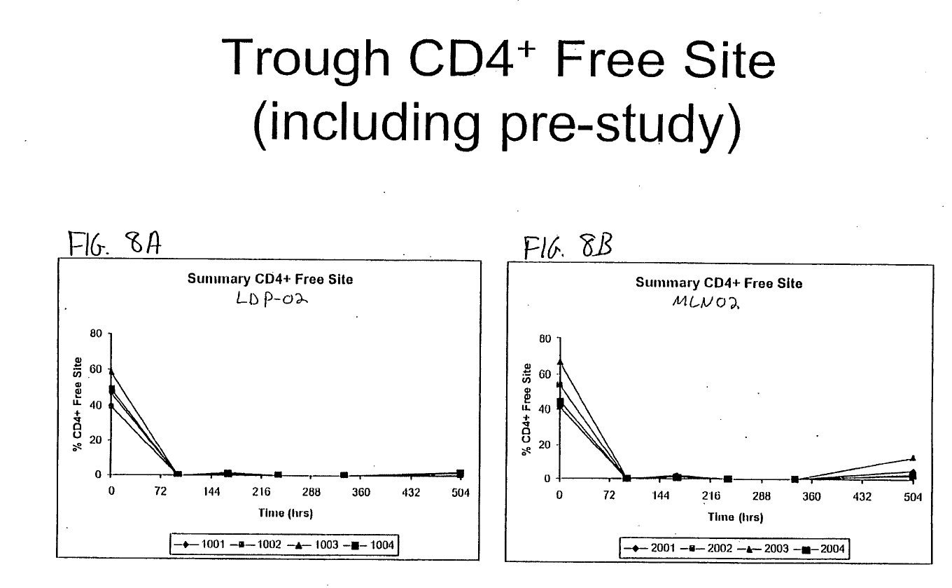

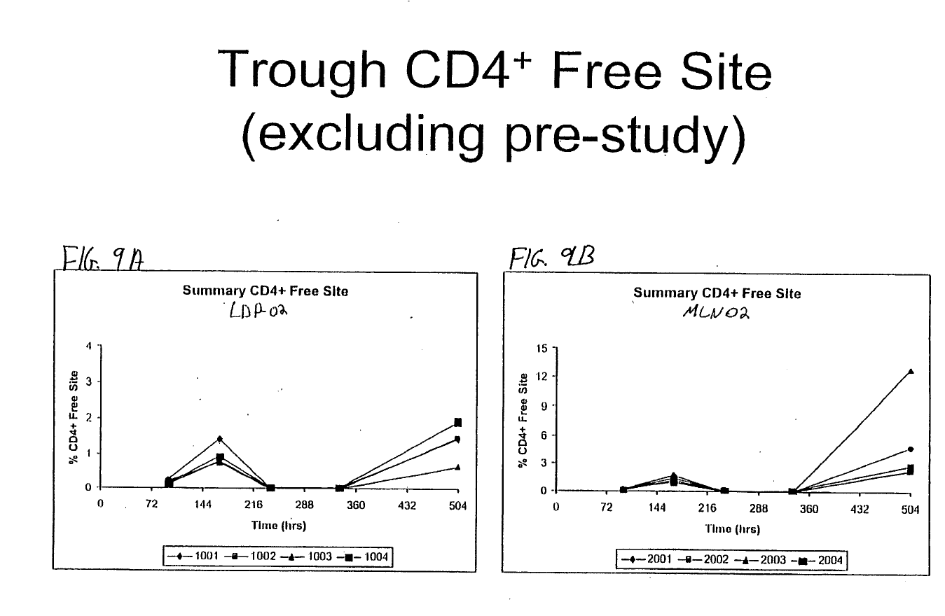

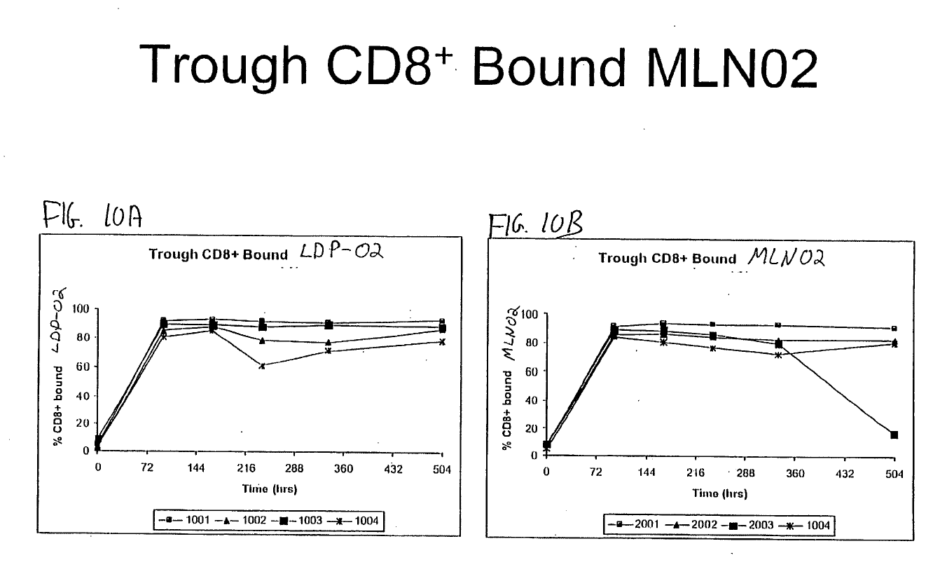

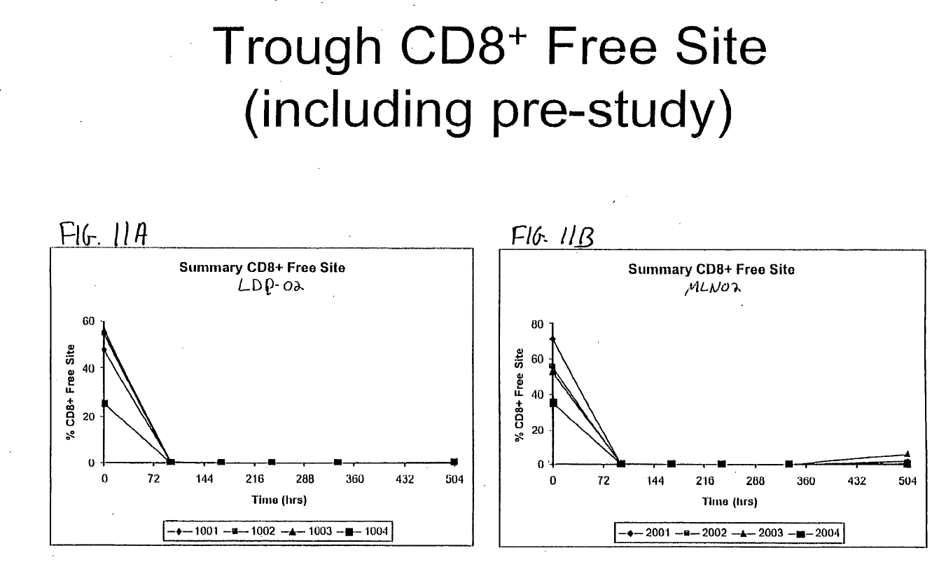

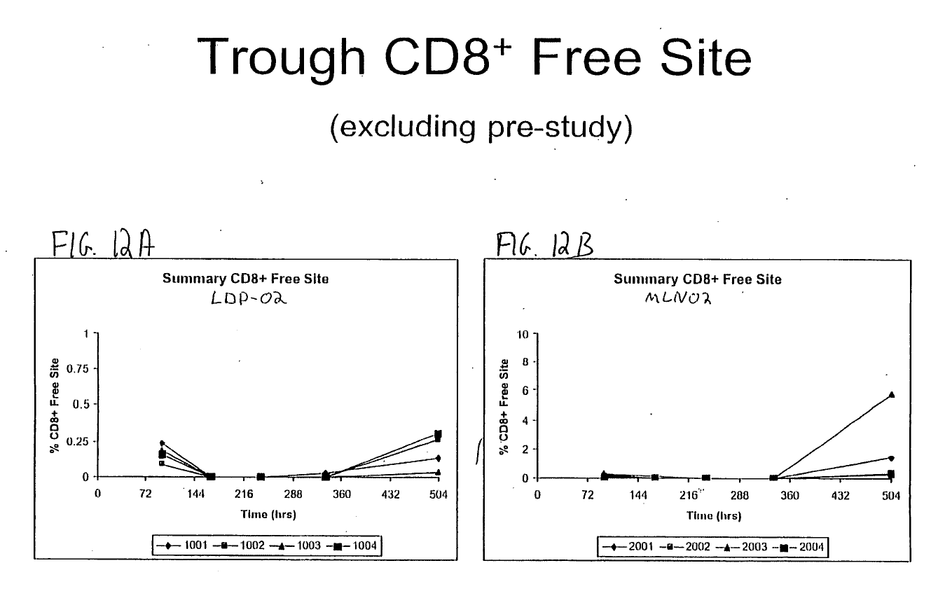

The PD studies provided qualitative measures of the CD4⁺ Free Site, CD4⁺ Bound LDP-02 or MLN02, CD8⁺ Free Site, and CD8⁺ Bound LDP-02 or MLN02. These studies showed a qualitative measure of LDP-02 or MLN02 bound to the cell surface, or measured the relative number of α4β7 sites not bound by LDP-02 or MLN02. The resulting data showed good saturation and receptor occupancy using either LDP-02 or MLN02. (FIGS. 7A-12B)

Additionally, it was determined that the potential for development of neutralizing antibodies (primate anti-humanized antibody antibodies) in cynomolgus monkeys existed. This was expected because the humanized antibody is a foreign protein (xenogenic) when administered to a cynomolgus monkey.

The results demonstrated that the pharmacodynamic and pharmacokinetic profiles of LDP-02 and MLN02 were about the same. There was little difference in the immunogenicity profile between LDP-02 and MLN02 in this primate study.

What is claimed is:

1. A humanized immunoglobulin that has binding specificity for α4β7 integrin, comprising a humanized heavy chain and a humanized light chain, wherein the humanized heavy chain comprises the amino acid sequence of amino acid residues 20-470 of SEQ ID NO:2, and the humanized light chain comprises the amino acid sequence of amino acid residues 20-238 of SEQ ID NO:4.

2. The humanized immunoglobulin of claim 1 wherein said humanized heavy chain is encoded by a nucleic acid comprising the nucleotide sequence of nucleotides 87-1433 of SEQ ID NO:1, and said humanized light chain is encoded by a nucleic acid comprising the nucleotide sequence of nucleotides 81-737 of SEQ ID NO:3.

3. The humanized immunoglobulin of claim 1 further comprising a detectable label.

4. An isolated nucleic acid encoding a humanized immunoglobulin heavy chain and a humanized immunoglobulin light chain, wherein said humanized immunoglobulin heavy chain and said humanized immunoglobulin light chain associate together to form a humanized immunoglobulin that has binding specificity for α4β7 integrin, and wherein said humanized immunoglobulin heavy chain comprises the amino acid sequence of amino acid residues 20-470 of SEQ ID NO:2, and said humanized immunoglobulin light chain comprises the amino acid sequence of amino acid residues 20-238 of SEQ ID NO:4.

5. The isolated nucleic acid of claim 4, wherein said isolated nucleic acid comprises a first nucleotide sequence that encodes said humanized heavy chain and a second nucleotide sequence that encodes said humanized light chain, wherein said first nucleotide sequence comprises the nucleotide sequence of nucleotides 87-1433 of SEQ ID NO:1, and said second nucleotide sequence comprises the nucleotide sequence of nucleotides 81-737 of SEQ ID NO:3.

6. A humanized immunoglobulin light chain comprising the amino acid sequence of amino acid residues 20-238 of SEQ ID NO:4.

7. An isolated nucleic acid encoding the humanized immunoglobulin light chain of claim 6.

8. A recombinant vector comprising a nucleic acid encoding the humanized immunoglobulin light chain of claim 6.

9. The recombinant vector of claim 8, wherein said nucleic acid comprises the nucleotide sequence of nucleotides 81-737 of SEQ ID NO: 3.

10. The recombinant vector of claim 8, further comprising a second nucleic acid encoding a humanized immunoglobulin heavy chain comprising the amino acid sequence of amino acid residues 20-470 of SEQ ID NO:2.

11. The recombinant vector of claim 10, wherein said second nucleic acid comprises the nucleotide sequence of nucleotides 87-1433 of SEQ ID NO:1.

12. A recombinant vector comprising a nucleic acid encoding a humanized immunoglobulin heavy chain and a humanized immunoglobulin light chain, wherein said humanized immunoglobulin heavy chain and said humanized immunoglobulin light chain associate together to form a humanized immunoglobulin that has binding specificity for α4β7 integrin, and wherein said humanized immunoglobulin heavy chain comprises the amino acid sequence of amino acid residues 20-470 of SEQ ID NO:2, and said humanized immunoglobulin light chain comprises the amino acid sequence of amino acid residues 20-238 of SEQ ID NO:4.

13. The recombinant vector of claim 12, wherein said isolated nucleic acid comprises a first nucleotide sequence that encodes said humanized heavy chain and a second nucleotide sequence that encodes said humanized light chain, wherein said first nucleotide sequence comprises the nucleotide sequence of nucleotides 87-1433 of SEQ ID NO:1, and said second nucleotide sequence comprises the nucleotide sequence of nucleotides 81-737 of SEQ ID NO:3.

14. A host cell comprising the isolated nucleic acid of claim 7.

15. A host cell comprising the recombinant vector of claim 8.

16. The host cell of claim 14, further comprising a second isolated nucleic acid encoding a humanized immunoglobulin heavy chain comprising the amino acid sequence of amino acid residues 20-470 of SEQ ID NO:2.

17. A host cell comprising the isolated nucleic acid of claim 4.

18. A host cell comprising the recombinant vector of claim 12.

19. The host cell of claim 17 wherein the isolated nucleic acid comprises a first nucleotide sequence that encodes a humanized heavy chain and a second nucleotide sequence that encodes a humanized light chain, wherein said first nucleotide sequence comprises the nucleotide sequence of nucleotides 87-1433 of SEQ ID NO:1, and said second nucleotide sequence comprises the nucleotide sequence of nucleotides 81-737 of SEQ ID NO:3.

20. A method of preparing a humanized immunoglobulin comprising maintaining a host cell of claim 16 under conditions appropriate for expression of a humanized immunoglobulin, whereby humanized immunoglobulin chains are expressed and a humanized immunoglobulin is produced.

21. A method of preparing a humanized immunoglobulin comprising maintaining a host cell of claim 17 under conditions appropriate for expression of a humanized immunoglobulin, whereby humanized immunoglobulin chains are expressed and a humanized immunoglobulin is produced.

22. A pharmaceutical composition comprising a humanized immunoglobulin of claim 1 and a physiologically acceptable vehicle or carrier.

23. A method of inhibiting the interaction of a first cell bearing α4β7 with a second cell bearing a ligand thereof, comprising contacting said first cell with an effective amount of the humanized immunoglobulin of claim 1.

24. A method for treating a disease associated with leukocyte infiltration of tissues expressing the molecule MAdCAM-1, comprising administering to a patient in need thereof an effective amount of the humanized immunoglobulin of claim 1.

25. The method of claim 24, wherein the disease is a disease associated with leukocyte infiltration of tissues as a result of binding of leukocytes expressing α4β7 integrin to gut-associated endothelium expressing the molecule MAdCAM-1.

26. A method for treating inflammatory bowel disease in a patient, comprising administering to the patient an effective amount of the humanized immunoglobulin of claim 1.

27. An immunoconjugate comprising the humanized antibody of claim 1.

28. The immunoconjugate of claim 27 further comprising a cytotoxic agent.

29. The immunoconjugate of claim 28 wherein said cytotoxic agent is selected from the group consisting of a maytansine, an auristatin, and a calicheamicin.


SEQUENCE LISTING (7 sequences)

SEQ ID NO: 1 - DNA, 1445 bp, Artificial Sequence
  humanized immunoglobulin heavy chain
gaattctcgagatcgatctcaccatgggatggagctgtatcatcctcttcttggtagcaacagctacaggtgtccactcccaggtgcaattggtgcagtctggggctgaggttaagaagcctggggcttcagtgaaggtgtcctgcaagggttctggctacaccttcaccagctactggatgcattgggtgaggcaggcgcctggccaacgtctagagtggatcggagagattgatccttctgagagtaatactaactacaatcaaaaattcaagggacgcgtcacattgactgtagacatttccgctagcacagcctacatggagctctccagcctgagatctgaggacactgcggtctactattgtgcaagagggggttacgacggatgggactatgctattgactactggggtcaaggcaccctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagagca

SEQ ID NO: 2 - protein, 470 aa, Artificial Sequence
  human immunoglobulin heavy chain
MGWSCIILFLVATATGVHSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 751 bp, Artificial Sequence
  humanized immunoglobulin light chain
gaattctcgagatcgatctcaccatgggatggagctgtatcatcctcttcttggtagcaacagctacaggtgtccactccgatgtagtgatgactcaaagtccactctccctgcctgtcacccctggagaaccagcttctatctcttgcaggtctagtcagagtcttgcaaagagttatgggaacacctatttgtcttggtacctgcagaagcctggccagtctccacagctcctcatctatgggatttccaacagattttctggggtgccagacaggttcagtggcagtggttcagggacagatttcacactcaagatctcgcgagtagaggctgaggacgtgggagtgtattactgcttacaaggtacacatcagccgtacacgttcggacaggggaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgttagtctagagcagc

SEQ ID NO: 4 - protein, 238 aa, Artificial Sequence
  humanized immunoglobulin light chain
MGWSCIILFLVATATGVHSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 239 aa, Artificial Sequence
  mature humanized light chain LDP-02
MKLPVRLLVLLLFWIPVSGGDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRADAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 107 aa, Homo sapiens [sequence omitted]

SEQ ID NO: 7 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC